(19) United States
(12) Patent Application Publication
(10) Pub. No.: US 2009/0305960 A1
(43) Pub. Date: Dec. 10, 2009

(54) Melanocortin Receptor-Specific Peptides for Treatment of Obesity / 669

(75) Inventors: Xin Chen, Furlong, PA (US); Shubh D. Sharma, Cranbury, NJ (US); Yi-Qun Shi, East Brunswick, NJ (US); Wei Yang, Edison, NJ (US)

Correspondence Address: MORGAN LEWIS & BOCKIUS LLP, 1111 PENNSYLVANIA AVENUE NW, WASHINGTON, DC, 20004, US

(73) Assignee: Palatin Technologies, Inc, Cranbury, NJ (US)

(21) Appl. No.: 12/480,420

(22) Filed: Jun. 8, 2009

Related U.S. Application Data
(60) Provisional application No. 61/059,903, filed on Jun. 9, 2008.

(51) Int. Cl. A61K 38/12; C07K 7/64; A61P 3/00

(52) U.S. Cl. 514/11; 530/321

(57) ABSTRACT

Melanocortin receptor-specific cyclic peptides of the formula

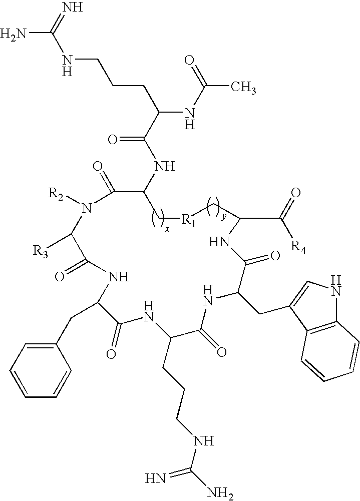

where R₁, R₂, R₃, R₄, x and y are as defined in the specification, compositions and formulations including the peptides of the foregoing formula, and methods of preventing, ameliorating or treating melanocortin receptor-mediated diseases, indications, conditions and syndromes, including obesity, diabetes, modulation of feeding behavior and related metabolic syndrome.

### CROSS-REFERENCE TO RELATED APPLICATION

This application claims priority to and the benefit of the filing of U.S. Provisional Patent Application Ser. No. 61/059,903 entitled “Melanocortin Receptor-Specific Peptides for Treatment of Obesity”, filed on Jun. 9, 2008, and the specification and claims thereof are incorporated herein by reference.

### BACKGROUND OF THE INVENTION

1. Field of the Invention (Technical Field)

The present invention relates to melanocortin receptor-specific cyclic peptides which may be used in the treatment of melanocortin receptor-mediated diseases, indications, conditions and syndromes.

2. Description of Related Art

The following discussion refers to a number of publications by author(s) and year of publication, and that due to recent publication dates certain publications are not to be considered as prior art vis-a-vis the present invention. Discussion of such publications herein is given for more complete background and is not to be construed as an admission that such publications are prior art for patentability determination purposes.

A family of melanocortin receptor types and subtypes have been identified, including melanocortin-1 receptors (MC1-R) expressed on normal human melanocytes and melanoma cells, melanocortin-2 receptors (MC2-R) for ACTH (adrenocorticotropin) expressed in cells of the adrenal gland, melanocortin-3 and melanocortin-4 receptors (MC3-R and MC4-R) expressed primarily in cells in the hypothalamus, mid-brain and brainstem, and melanocortin-5 receptors (MC5-R), expressed in a wide distribution of peripheral tissues.

Significant work has been done in determining the structure of melanocortin receptors, including both the nucleic acid sequences encoding for the receptors and the amino acid sequences constituting the receptors. MC4-R is a G protein-coupled, 7-transmembrane receptor that is believed to be expressed primarily in the brain.

Peptides specific for MC4-R, and secondarily peptides specific for MC3-R, are believed to be useful in regulation of mammalian energy homeostasis, including use as agents for attenuating food intake and body weight gain. MC4-R agonist peptides are believed to be useful for treating sexual dysfunction, including male erectile dysfunction, and for decreasing food intake and body weight gain, such as for treatment of obesity. MC4-R agonist peptides, as well as MC3-R agonist peptides, may also be employed for decreasing voluntary ethanol consumption, treatment of drug addictions, and the like. MC4-R agonist peptides, as well as MC1-R and MC3-R agonist peptides, may further be employed for treatment of circulatory shock, ischemia, hemorrhagic shock, inflammatory diseases and related diseases, indications, conditions and syndromes. MC4-R antagonists, by contrast, are believed to be useful for weight gain aid, such as for use in treatment of cachexia, sarcopenia, wasting syndrome or disease, and anorexia. Such peptides may also be employed for treatment of depression and related disorders.

Melanocortin receptor-specific peptides include cyclic α-melanocyte-stimulating hormone (“α-MSH”) analog peptides such as Ac-Nle-cyclo(-Asp-His-D-Phe-Arg-Trp-Lys)-NH₂ (SEQ ID NO:1) (See U.S. Pat. Nos. 5,674,839 and 5,576,290) and Ac-Nle-cyclo(-Asp-His-D-Phe-Arg-Trp-Lys)-OH (SEQ ID NO:2) (See U.S. Pat. Nos. 6,579,968 and 6,794,489). These and other melanocortin receptor-specific peptides generally contain the central tetrapeptide sequence of native α-MSH, His⁶-Phe⁷-Arg⁸-Trp⁹ (SEQ ID NO:3), or a mimetic or variation thereof, including the substitution of D-Phe for Phe⁷. Other peptides or peptide-like compounds asserted to be specific for one or more melanocortin receptors are disclosed in U.S. Pat. Nos. 5,731,408, 6,054,556, 6,350,430, 6,476,187, 6,600,015, 6,613,874, 6,693,165, 6,699,873, 6,887,846, 6,951,916, 7,008,925, and 7,176,279; in U.S. published patent application Publication Nos. 2001/0056179, 2002/0143141, 2003/0064921, 2003/0105024, 2003/0212002, 2004/0023859, 2005/0130901, 2005/0187164, 2005/0239711, 2006/0105951, 2006/0111281, 2006/0293223, 2007/0027091, 2007/0105759, 2007/0123453, 2007/0244054, and 2008/0039387; and in international patent applications nos. WO 98/27113, WO 99/21571, WO 00/05263, WO 99/54358, WO 00/35952, WO 00/58361, WO 01/30808, WO 01/52880, WO 01/74844, WO 01/85930, WO 01/90140, WO 02/18437, WO 02/26774, WO 03/006604, WO 2004/046166, WO 2005/000338, WO 2005/000339, WO 2005/000877, WO 2005/030797, WO 2005/060985, WO2006/048449, WO 2006/048450, WO 2006/048451, WO 2006/048452, WO 2006/097526, WO 2007/008684, WO 2007/008704, and WO 2007/009894.

Notwithstanding the intense scientific and pharmaceutical interest in melanocortin receptor-specific peptides, evidenced by numerous articles in the scientific literature and numerous patent applications and issued patents, no melanocortin receptor-specific peptide has been approved as a drug for any therapeutic indication. Indeed, there are no reports of any melanocortin receptor-specific peptide for any therapeutic indication having advanced past Phase II clinical trials. There remains a significant and substantial need for melanocortin receptor-specific peptides for use in pharmaceutical applications. It is against this background that the present invention was made.

### BRIEF SUMMARY OF THE INVENTION

In one aspect, the present invention provides a cyclic peptide of formula (I):

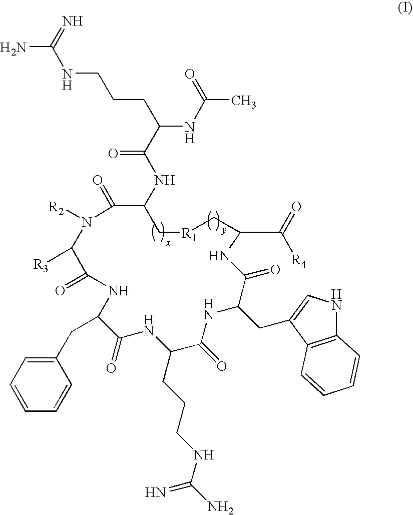

including all enantiomers, stereoisomers or diastereoisomers thereof, or a pharmaceutically acceptable salt of any of the foregoing,

wherein:
R₁ is —C(═O)—NH— or —NH—C(═O)—;
R₂ is —H or is —CH₂—, and if it is —CH₂— forms a pyrrolidine ring with R₃ of the structure

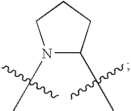

R₃ is —(CH₂)₂—, and if it is —(CH₂)₂— forms a pyrrolidine ring with R₂, or is

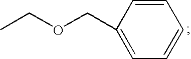

R₄ is —OH or —NH₂; and
if R₁ is —C(═O)—NH—, then x is 1 and y is 4 or x is 2 and y is 3, and if R₁ is —NH—C(═O)—, then x is 3 and y is 2.

The invention thus includes a cyclic peptide of formula (II):

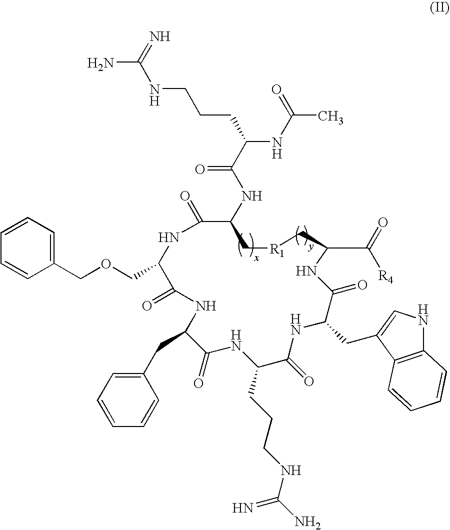

or a pharmaceutically acceptable salt thereof. This includes the cyclic peptides:

(SEQ ID NO:4)

Ac-Arg-cyclo(Glu-Ser(Bzl)-D-Phe-Arg-Trp-Orn)-OH;

(SEQ ID NO:5)

Ac-Arg-cyclo(Orn-Ser(Bzl)-D-Phe-Arg-Trp-Glu)-OH;

(SEQ ID NO:6)

Ac-Arg-cyclo(Asp-Ser(Bzl)-D-Phe-Arg-Trp-Lys)-OH;
and

(SEQ ID NO:7)

Ac-Arg-cyclo(Asp-Ser(Bzl)-D-Phe-Arg-Trp-Lys)-NH₂.

The invention further includes a cyclic peptide of formula (III):

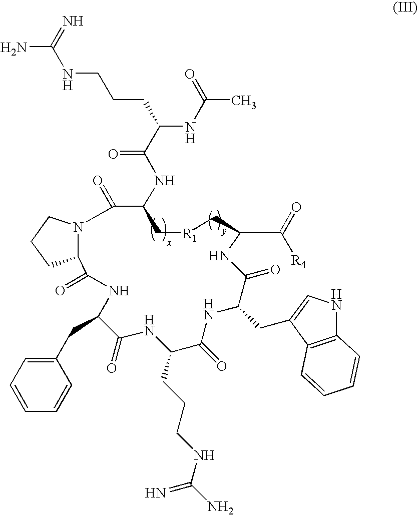

or a pharmaceutically acceptable salt thereof. This includes the cyclic peptides:

(SEQ ID NO:8)

Ac-Arg-cyclo(Asp-Pro-D-Phe-Arg-Trp-Lys)-NH₂;

(SEQ ID NO:9)

Ac-Arg-cyclo(Orn-Pro-D-Phe-Arg-Trp-Glu)-NH₂;
and

(SEQ ID NO:10)

Ac-Arg-cyclo(Asp-Pro-D-Phe-Arg-Trp-Lys)-OH.

In another aspect, the present invention provides a melanocortin receptor-specific peptide-based pharmaceutical composition for use in treatment of melanocortin receptor-mediated diseases, indications, conditions and syndromes, including obesity, diabetes and related metabolic syndrome.

In another aspect, the present invention provides a peptide-based melanocortin receptor-specific pharmaceutical, wherein the peptide is a selective MC4-R ligand, for use in treatment of obesity, diabetes, related metabolic syndrome, modulation of feeding behavior and other MC4-R associated disorders.

In another aspect, the present invention provides peptides which are specific for MC4-R and which are partial agonists.

In another aspect, the present invention provides MC4-R agonist peptides which do not, or do not substantially, induce a sexual response in a mammal, including not inducing a penile erection in a male. Without being bound by any theory, it is believed that peptides that are partial agonists in the assay for agonist activity described herein (i.e., compounds having intrinsic activity of from 10% to 70%) will provide no or a reduced level of sexual effects resulting from MC4-R activation, including penile erections in assays as described herein, compared to compounds that are full agonists in the same assay. Sexual effects are considered unwanted side-effects for treatment of obesity, diabetes or related metabolic syndrome.

In another aspect, the present invention provides peptides which are specific for MC4-R and which are at least twenty-fold less specific for MC1-R.

In another aspect, the present invention provides a peptide-based melanocortin receptor-specific pharmaceutical for use in treatment of obesity, modulation of feeding behavior and other energy homeostasis disorders.

In another aspect, the present invention provides a melanocortin receptor-specific pharmaceutical for use in treatment wherein administration of the treatment is via nasal administration.

According to one embodiment of the present invention, there are provided cyclic peptides that are MC4-R specific partial agonists, such cyclic peptides characterized in part by having an Arg residue in the first position and outside the cyclic portion of the peptide, and have a Pro or Ser(Bzl) substituted for His in the His-D-Phe-Arg-Trp sequence.

According to another embodiment of the present invention, there are provided MC4-R specific cyclic peptides for use in treatment of eating disorders which, because of increased efficacy at low doses, may be administered by delivery systems other than art conventional intravenous, subcutaneous or intramuscular injection, including but not limited to oral delivery systems, nasal delivery systems and mucous membrane delivery systems.

Other aspects and novel features, and the further scope of applicability of the present invention will be set forth in part in the detailed description to follow, and in part will become apparent to those skilled in the art upon examination of the following, or may be learned by practice of the invention. The aspects of the invention may be realized and attained by means of the instrumentalities and combinations particularly pointed out in the appended claims.

### DETAILED DESCRIPTION OF THE INVENTION

### 1.0 Definitions

Before proceeding with the description of the invention, certain terms are defined as set forth herein.

In the sequences given for the peptides according to the present invention, the amino acid residues have their conventional meaning as given in Chapter 2400 of the Manual of Patent Examining Procedure, 8^{th} Ed., published by the United States Patent and Trademark Office. Thus, “Asp” is aspartic acid, “His” is histidine, “Phe” is phenylalanine, “Pro” is proline, “Arg” is arginine, “Trp” is tryptophan, and “Lys” is lysine, and so on. It is to be understood that “D” isomers are designated by a “D-” before the three letter code or amino acid name, such that for example D-Phe is D-phenylalanine. Amino acid residues not encompassed by the foregoing have the following definitions:

| Abbreviation | Common Name | Side Chain Structure |
|---|---|---|
| Orn | ornithine | 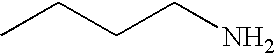 |
| Ser(Bzl) | O-benzyl-serine | 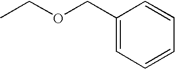 |

The term “Ac” means the acetyl group CH₃—C(═O)—.

An “amide” includes compounds that have a trivalent nitrogen attached to a carbonyl group (—C(═O)—NH₂), such as for example methylamide, ethylamide, propylamide, and the like.

An “amine” includes compounds that contain an amino group (—NH₂).

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions utilized in the present invention encompass any composition made by admixing an active ingredient and one or more pharmaceutically acceptable carriers.

By a melanocortin receptor “agonist” is meant an endogenous substance, drug substance or compound, including a compound such as the cyclic peptides of the present invention, which can interact with a melanocortin receptor and initiate a pharmacological response, including but not limited to adenyl cyclase activation, characteristic of the melanocortin receptor.

By “α-MSH” is meant the peptide Ac-Ser-Tyr-Ser-Met-Glu-His-Phe-Arg-Trp-Gly-Lys-Pro-Val-NH₂ (SEQ ID NO:12) and analogs and homologs thereof, including without limitation NDP-α-MSH.

By “NDP-α-MSH” is meant the peptide Ac-Ser-Tyr-Ser-Nle-Glu-His-D-Phe-Arg-Trp-Gly-Lys-Pro-Val-NH₂ (SEQ ID NO:13) and analogs and homologs thereof.

By “EC₅₀” is meant the molar concentration of an agonist, including a partial agonist, which produced 50% of the maximum possible response for that agonist. By way of example, a test compound which, at a concentration of 72 nM, produces 50% of the maximum possible response for that compound as determined in a cAMP assay in an MC4-R cell expression system has an EC₅₀ of 72 nM. Unless otherwise specified, the molar concentration associated with an EC₅₀ determination is in nanomoles per liter (nM).

By “Ki (nM)” is meant the equilibrium inhibitor dissociation constant representing the molar concentration of a competing compound that binds to half the binding sites of a receptor at equilibrium in the absence of radioligand or other competitors. In general, the numeric value of the Ki is inversely correlated to the affinity of the compound for the receptor, such that if the Ki is low, the affinity is high. Ki may be determined using the equation of Cheng and Prusoff (Cheng Y., Prusoff W. H., Biochem. Pharmacol. 22: 3099-3108, 1973):

$${K}_{i} = \frac{{EC}_{50}}{1 + \frac{[ ligand ]}{{K}_{D}}}$$

where “ligand” is the concentration of radioligand and K_{D} is an inverse measure of receptor affinity for the radioligand which produces 50% receptor occupancy by the radioligand. Unless otherwise specified, the molar concentration associated with a Ki determination is in nM. Ki may be expressed in terms of specific receptors (e.g., MC1-R, MC3-R, MC4-R or MC5-R) and specific ligands (e.g., α-MSH or NDP-α-MSH).

By “inhibition” is meant the percent attenuation, or decrease in receptor binding, in a competitive inhibition assay compared to a known standard. Thus, by “inhibition at 1 μM (NDP-α-MSH)” is meant the percent decrease in binding of NDP-α-MSH by addition of a determined amount of the compound to be tested, such as 1 μM of a test compound, such as under the assay conditions hereafter described. By way of example, a test compound that does not inhibit binding of NDP-α-MSH has a 0% inhibition, and a test compound that completely inhibits binding of NDP-α-MSH has a 100% inhibition. Typically, as described hereafter, a radio assay is used for competitive inhibition testing, such as with I¹²⁵-labeled NDP-α-MSH, or a lanthanide chelate fluorescent assay, such as with Eu-NDP-α-MSH. However, other methods of testing competitive inhibition are known, including use of label or tag systems other than radioisotopes, and in general any method known in the art for testing competitive inhibition may be employed in this invention. It may thus be seen that “inhibition” is one measure to determine whether a test compound attenuates binding of α-MSH to melanocortin receptors.

By “binding affinity” is meant the ability of a compound or drug to bind to its biological target, expressed herein as Ki (nM).

By “intrinsic activity” is meant the maximal functional activity achievable by a compound in a specified melanocortin receptor expressing cell system, such as the maximal stimulation of adenylyl cyclase. The maximal stimulation achieved by α-MSH or NDP-α-MSH is designated as an intrinsic activity of 1.0 (or 100%) and a compound capable of stimulating half the maximal activity that of α-MSH or NDP-α-MSH is designated as having an intrinsic activity of 0.5 (or 50%). A compound of this invention that under assay conditions described herein has an intrinsic activity of 0.7 (70%) or higher is classified as an agonist, a compound with intrinsic activity between 0.1 (10%) and 0.7 (70%) is classified as a partial agonist, and a compound with intrinsic activity below 0.1 (10%) is classified as inactive or having no intrinsic activity. In one aspect, the cyclic peptides of the present invention may generally be characterized as a partial agonist at MC4-R with respect to α-MSH or NDP-α-MSH.

In general, “functional activity” is a measure of the signaling of a receptor, or measure of a change in receptor-associated signaling, such as a melanocortin receptor, and in particular MC4-R or hMC4-R, upon activation by a compound. Melanocortin receptors initiate signal transduction through activation of heterotrimeric G proteins. In one aspect, melanocortin receptors signal through Gαₛ, which catalyzes production of cAMP by adenylyl cyclase. Thus determination of stimulation of adenylyl cyclase, such as determination of maximal stimulation of adenylyl cyclase, is one measure of functional activity, and is the primary measure exemplified herein. However, it is to be understood that alternative measures of functional activity may be employed in the practice of this invention, and are specifically contemplated and included within the scope of this invention. Thus, in one example intracellular free calcium may be measured, such as reported by and using the methods disclosed in Mountjoy K. G. et al., Melanocortin receptor-medicated mobilization of intracellular free calcium in HEK293 cells. Physiol Genomics 5:11-19, 2001, or Kassack M. U. et al., Functional screening of G protein-coupled receptors by measuring intracellular calcium with a fluorescence microplate reader. Biomol Screening 7:233-246, 2002. It is also possible to measure activation by measurement of the production of inositol triphosphate or diacylglycerol from phosphatidylinositol 4,5-biphosphate, such as by use of radioassays. Yet another measure of functional activity is receptor internalization, resulting from activation of regulatory pathways, such as using the methods disclosed in Nickolls S. A. et al., Functional selectivity of melanocortin 4 receptor peptide and nonpeptide agonists: evidence for ligand specific conformational states. J Pharm Exper Therapeutics 313:1281-1288, 2005. Yet another measure of functional activity is the exchange, and exchange rate, of nucleotides associated with activation of a G protein receptor, such as the exchange of GDP (guanosine diphosphate) for GTP (guanosine triphosphase) on the G protein α subunit, which may be measured by any number of means, including a radioassay using guanosine 5′-(γ-[³⁵S]thio)-triphosphate, as disclosed in Manning D. R., Measures of efficacy using G proteins as endpoints: differential engagement of G proteins through single receptors. Mol Pharmacol 62:451-452, 2002. Various gene-based assays have been developed for measuring activation of G-coupled proteins, such as those disclosed in Chen W. et al., A colorimetric assay from measuring activation of Gs- and Gq-coupled signaling pathways. Anal Biochem 226:349-354, 1995; Kent T. C. et al., Development of a generic dual-reporter gene assay for screening G-protein-coupled receptors. Biomol Screening, 5:437-446, 2005; or Kotarsky K. et al., Improved receptor gene assays used to identify ligands acting on orphan seven-transmembrane receptors. Pharmacology & Toxicology 93:249-258, 2003. The calorimetric assay of Chen et al. has been adapted for use in measuring melanocortin receptor activation, as disclosed in Hruby V. J. et al., Cyclic lactam α-melanocortin analogues of Ac-Nle⁴-cyclo[Asp⁵, D-Phe⁷, Lys¹⁰]α-melanocyte-stimulating hormone-(4-10)-NH₂ with bulky aromatic amino acids at position 7 shows high antagonist potency and selectivity at specific melanocortin receptors. J Med Chem 38:3454-3461, 1995. In general, functional activity may be measured by any method, including methods of determining activation and/or signaling of a G-coupled receptor, and further including methods which may be hereafter developed or reported. Each of the foregoing articles, and the methods disclosed therein, is incorporated here by reference as if set forth in full.

The terms “treat,” “treating” and “treatment,” as used herein, contemplate an action that occurs while a patient is suffering from the specified disease or disorder, which reduces the severity of the disease or disorder.

As used herein, the term “therapeutically effective amount” means the amount of a compound including a peptide of the invention that will elicit a biological or medical response in the mammal that is being treated by a medical doctor or other clinician.

As used herein, the term “prophylactically effective” or “preventive” means the amount of a compound including a peptide of the invention that will prevent or inhibit affliction or mitigate affliction of a mammal with a medical condition that a medical doctor or other clinician is trying to prevent, inhibit, or mitigate before a patient begins to suffer from the specified disease or disorder.

The term “obesity” means the condition of excess body fat (adipose tissue), including by way of example in accordance with the National Institutes of Health Federal Obesity Clinical Guidelines for adults, whereby body mass index calculated by dividing body mass in kilograms by height in meters squared is equal to or greater than twenty-five (25), and further including an overweight condition and comparable obesity and overweight condition in children.

The term “diabetes” includes Type 1 Diabetes, which is insulin-dependent diabetes mellitus as diagnosed according to criteria published in the Report of the Expert Committee on the Diagnosis and Classification of Diabetes Mellitus (Diabetes Care, Vol. 24, Supp. 1, January 2001) whereby fasting plasma glucose level is greater than or equal to 126 milligrams per deciliter and for which the primary cause is pancreatic beta cell destruction, Type 2 Diabetes, which is non-insulin-dependent diabetes mellitus as diagnosed according to criteria published in the Report of the Expert Committee on the Diagnosis and Classification of Diabetes Mellitus whereby fasting plasma glucose level is greater than or equal to 126 milligrams per deciliter, and latent autoimmune diabetes mellitus of the adult (LADA).

The term “metabolic syndrome” refers to metabolic disorders, particularly glucose and lipid regulatory disorders, including insulin resistance and defective secretion of insulin by pancreatic beta cells, and may further include conditions and states such as abdominal obesity, dyslipidemia, hypertension, glucose intolerance or a prothrombotic state, and which may further result in disorders such as hyperlipidemia, obesity, diabetes, insulin resistance, glucose intolerance, hyperglycemia, and hypertension.

### 2.0 Clinical Indications and Utility

The compositions and methods disclosed herein can be used for both medical applications and animal husbandry or veterinary applications. Typically, the methods are used in humans, but may also be used in other mammals. The term “patient” is intended to denote a mammalian individual, and is so used throughout the specification and in the claims. The primary applications of the present invention involve human patients, but the present invention may be applied to laboratory, farm, zoo, wildlife, pet, sport or other animals. Clinical indications and specific utilities include the following:

2.1 Obesity, Diabetes and Related Metabolic Syndrome

Peptides of formula (I), and in particular formula (II) or (III), have been found to be ligands of the MC4 receptor. In particular, peptides of formula (I) are believed to be useful in treating diseases, disorders and/or conditions responsive to modulation of the MC4-R function, more particularly activation of the MC4-R, i.e. diseases, disorders and/or conditions which would benefit from agonism (including full or partial agonism) at the MC4-R, including energy homeostasis and metabolism related (such as diabetes, in particular type 2 diabetes; dyslipidemia; fatty liver; hypercholesterolemia; hypertriglyceridemia; hyperuricacidemia; impaired glucose tolerance; impaired fasting glucose; insulin resistance syndrome; and metabolic syndrome), food intake related (such as hyperphagia; binge eating; bulimia; and compulsive eating) and/or energy balance and body weight related diseases, disorders and/or conditions, more particularly such diseases, disorders and conditions characterized by excess body weight and/or excess food intake.

Peptides of formula (I), and in particular formula (II) or (III), are particularly believed to be useful for treatment of body weight related diseases, disorders and/or conditions characterized by excess body weight, including obesity and overweight (by promotion of weight loss, maintenance of weight loss, and/or prevention of weight gain, including medication-induced weight gain or weight gain subsequent to cessation of smoking), and diseases, disorders and/or conditions associated with obesity and/or overweight, such as insulin resistance; impaired glucose tolerance; type 2 diabetes; metabolic syndrome; dyslipidemia (including hyperlipidemia); hypertension; heart disorders (e.g. coronary heart disease, myocardial infarction); cardiovascular disorders; non-alcoholic fatty liver disease (including non-alcoholic steatohepatitis); joint disorders (including secondary osteoarthritis); gastroesophageal reflux; sleep apnea; atherosclerosis; stroke; macro and micro vascular diseases; steatosis (e.g. in the liver); gall stones; and gallbladder disorders.

It will be understood that there are medically accepted definitions of obesity and overweight. A patient may be identified by, for example, measuring body mass index (BMI), which is calculated by dividing weight in kilograms by height in meters squared, and comparing the result with the definitions. The recommended classifications for BMI in humans, adopted by the Expert Panel on the Identification, Evaluation and Treatment of Overweight and Obesity in Adults, and endorsed by leading organizations of health professionals, are as follows: underweight <18.5 kg/m², normal weight 18.5-24.9 kg/m², overweight 25-29.9 kg/m₂, obesity (class 1) 30-34.9 kg/m², obesity (class 2) 35-39.9 kg/m², extreme obesity (class 3)≧40 kg/m² (Practical Guide to the Identification, Evaluation, and Treatment of Overweight and Obesity in Adults, The North American Association for the Study of Obesity (NAASO) and the National Heart, Lung and Blood Institute (NHLBI) 2000). Modifications of this classification may be used for specific ethnic groups. Another alternative for assessing overweight and obesity is by measuring waist circumference. There are several proposed classifications and differences in the cutoffs based on ethnic group. For instance, according to the classification from the International Diabetes Federation, men having waist circumferences above 94 cm (cut off for europids) and women having waist circumferences above 80 cm (cut off for europids) are at higher risk of diabetes, dyslipidemia, hypertension and cardiovascular diseases because of excess abdominal fat. Another classification is based on the recommendation from the Adult Treatment Panel III where the recommended cut-offs are 102 cm for men and 88 cm for women. However, the peptides of Formula I may also be used for reduction of self-diagnosed overweight and for decreasing the risk of becoming obese due to life style, genetic considerations, heredity and/or other factors.

It is believed that peptides of formula (I), and in particular formula (II) or (III), upon administration to an animal, including man, will reduce food intake, body weight and/or body weight gain in that animal.

Without being bound by any theory, it is believed that peptides of formula (I), and in particular formula (II) or (III), act by modulating appetite and/or satiety, increasing metabolic rate, reducing intake of and/or craving for fat and/or carbohydrates.

Without being bound by any theory, it is also believed that peptides of formula (I), and in particular formula (II) or (III), act by enhancing glucose tolerance and/or decreasing insulin resistance. It is therefore believed that peptides of formula (I) can be useful also for treatment of type 2 diabetes in underweight and normal weight individuals as well as in overweight and obese individuals.

Peptides of the invention might also be useful for (i) occlusive, haemorrhagic, traumatic or surgical organ and/or tissue damage, such as myocardial infarction and stroke, (ii) haemorrhagic or cardiogenic shock, or (iii) male and female sexual dysfunctions, such as male erectile dysfunction.

According to a further aspect of the invention, there is provided a peptide of formula (I), and in particular formula (II) or (III), as previously defined for use as a medicament.

In another aspect, the invention provides the use of a peptide of formula (I), and in particular formula (II) or (III), for treatment of diseases, disorders and/or conditions responsive to modulation of the MC4-R, such as diseases, disorders and/or conditions responsive to activation of the MC4-R, in particular energy homeostasis and metabolism related (e.g. diabetes), food intake related and/or energy balance and body weight related diseases, disorders and/or conditions, including obesity, overweight and diseases, disorders and/or conditions associated with obesity and/or overweight, such as type 2 diabetes and metabolic syndrome.

In a further aspect, the invention provides the use of a peptide of formula (I), and in particular formula (II) or (III), in the preparation of a medicament for treatment of diseases, disorders and/or conditions responsive to modulation of the MC4-R, such as activation of the MC4-R, in particular energy homeostasis and metabolism related (e.g. diabetes), food intake related and/or energy balance and body weight related diseases, disorders and/or conditions, including obesity, overweight and diseases, disorders and/or conditions associated with obesity and/or overweight, such as type 2 diabetes and metabolic syndrome.

2.2 Addiction Related Diseases, Indications, Conditions and Syndromes

In one aspect, one or more of the present peptides may be employed for inhibiting alcohol consumption, or for reducing alcohol consumption, or for treating or preventing alcoholism, or for treating or preventing alcohol abuse, or for treating or preventing alcohol-related disorders. In another related aspect, one or more of the present peptides may be employed for inhibiting consumption of drugs of abuse, or for reducing consumption of drugs of abuse, or for treating or preventing drug abuse, or for treating or preventing drug abuse-related disorders. Drugs of abuse are typically controlled substances. These include controlled naturally derived drugs such as heroin, morphine, opium, cocaine, marijuana and the like, as well as synthetically made drugs such as Vicodin®, Lortab®, Lorcet®, Percocet®, Percodan®, Tylox®, hydrocodone, OxyContin®., methadone, tramadol, various methamphetamines, and other tranquilizers, stimulants, or sedatives known to be abused, as well as drugs for which there is no established pharmaceutical utility, such as ecstasy, LSD, or PCP.

### 3.0 Combination Therapy for Certain Indications

The peptides, compositions and methods of the present invention may be used for treatment of any of the foregoing diseases, indications, conditions or syndromes, or any disease, indication, condition or syndrome which is melanocortin receptor mediated, by administration in combination with one or more other pharmaceutically active peptides. Such combination administration may be by means of a single dosage form which includes both a peptide of the present invention and one more other pharmaceutically active compounds, such single dosage form including a tablet, capsule, spray, inhalation powder, injectable liquid or the like. Alternatively, combination administration may be by means of administration of two different dosage forms, with one dosage form containing a peptide of the present invention, and the other dosage form including another pharmaceutically active compound. In this instance, the dosage forms may be the same or different. Without meaning to limit combination therapies, the following exemplifies certain combination therapies which may be employed.

3.1 Combination Therapy for Obesity, Diabetes and Related Metabolic Syndrome

It is possible and contemplated to use peptides of the present invention in combination with other drugs or agents for treatment of various weight and feeding-related disorders. Peptides of the present invention may be employed for decreasing food intake and/or body weight in combination with any other agent or drug heretofore employed as a diet aid, or for decreasing food intake and/or body weight. Peptides of the present invention may further be employed for increasing food intake and/or body weight in combination with any other agent or drug heretofore employed for increasing food intake and/or body weight.

Drugs that reduce energy intake include, in part, various pharmacological agents, referred to as anorectic drugs, which are used as adjuncts to behavioral therapy in weight reduction programs. Classes of anorectic drugs include, but are not limited to, noradrenergic and serotonergic agents. Noradrenergic medications may be described as those medications generally preserving the anorectic effects of amphetamines but with weaker stimulant activity. The noradrenergic drugs, except phenylpropanolamine, generally act through a centrally mediated pathway in the hypothalamus that causes anorexia. Phenylpropanolamine, a racemic mixture of norephedrine esters, causes a release of norepinephrine throughout the body and stimulates hypothalamic adrenoreceptors to reduce appetite.

Suitable noradrenergic agents include, but are not limited to, diethylpropion such as TENUATE™ (1-propanone, 2-(diethylamino)-1-phenyl-, hydrochloride) commercially available from Merrell; mazindol (or 5-(p-chlorophenyl)-2,5-dihydro-3H-imidazo[2,1-a]isoindol-5-ol) such as SANOREX™ commercially available from Novartis or MAZANOR™ commercially available from Wyeth Ayerst; phenylpropanolamine (or Benzenemethanol, alpha-(1-aminoethyl)-, hydrochloride); phentermine (or Phenol, 3-[[4,5-duhydro-1H-imidazol-2-yl)ethyl](4-methylphenyl)amino], monohydrochloride) such as ADIPEX-P™ commercially available from Lemmon, FASTIN™ commercially available from Smith-Kline Beecham and Ionamin™ commercially available from Medeva; phendimetrazine (or (2S,3S)-3,4-Dimethyl-2phenylmorpholine L-(+)-tartrate (1:1)) such as METRA™ commercially available from Forest, PLEGINE™ commercially available from Wyeth-Ayerst; PRELU-2™ commercially available from Boehringer Ingelheim, and STATOBEX™ commercially available from Lemmon; phendamine tartrate such as THEPHORIN™ (2,3,4,9-Tetrahydro-2-methyl-9-phenyl-1H-indenol[2,1-c]pyridine L-(+)-tartrate (1:1)) commercially available from Hoffmann-LaRoche; methamphetamine such as DESOXYN™ Tablets ((S)—N, (alpha)-dimethylbenzeneethanamine hydrochloride) commercially available from Abbott; and phendimetrazine tartrate such as BONTRIL™ Slow-Release Capsules (-3,4-Dimethyl-2-phenylmorpholine Tartrate) commercially available from Amarin.

Suitable serotonergic agents include, but are not limited to, sibutramine such as MERIDIA™ capsules (a racemic mixture of the (+) and (−) enantiomers of cyclobutanemethanamine, 1-(4-chlorophenyl)-N,N-dimethyl-(alpha)-(2-methylpropyl)-, hydrochloride, monohydrate) commercially available from Knoll, fenfluramine such as Pondimin™ (Benzeneethanamine, N-ethyl-alpha-methyl-3-(trifluoromethyl)-, hydrochloride) commercially available from Robbins; dexfenfluramine such as Redux™ (Benzeneethanamine, N-ethyl-alpha-methyl-3-(trifluoromethyl)-, hydrochloride) commercially available from Interneuron. Fenfluramine and dexfenfluramine stimulate release of serotonin and inhibit its reuptake. Sibutramine inhibits the reuptake of serotonin, norepinephrine and dopamine, but does not stimulate secretion of serotonin.

Other serotonergic agents useful with the practice of the present invention include, but are not limited to, certain auoretic gene 5HT1a inhibitors (brain, serotonin) such as carbidopa and benserazide as disclosed by U.S. Pat. No. 6,207,699 which is incorporated herein by reference; and certain neurokinin 1 receptor antagonist and selective serotonin reuptake inhibitors including fluoxetine, fluvoxamine, paroxtine, sertraline and other useful compounds as disclosed by U.S. Pat. No. 6,162,805 which is incorporated herein by reference. Other potential agents that may be employed include, for example, 5HT2c agonists, such as lorcaserin hydrochloride under development by Arena Pharmaceuticals.

Other useful compounds for reducing energy intake include, but are not limited to, certain aryl-substituted cyclobutylalkylamines as disclosed by U.S. Pat. No. 6,127,424 which is incorporated herein by reference; certain trifluoromethylthiophenylethylamine derivatives as disclosed by U.S. Pat. No. 4,148,923 which is incorporated herein by reference; certain compounds as disclosed by U.S. Pat. No. 6,207,699 which is incorporated herein by reference; certain kainite or AMPA receptor antagonists as disclosed by U.S. Pat. No. 6,191,117 which is incorporated herein by reference; certain neuropeptide receptor subtype 5 as disclosed by U.S. Pat. No. 6,140,354 which is incorporated herein by reference; and certain alpha-blocking agents as disclosed by U.S. Pat. No. 4,239,763 which is incorporated herein by reference.

In another aspect, one or more peptides of the present invention are administered with an opioid antagonist. The opioid antagonist may antagonize a mammalian μ-opioid receptor, preferably a human μ-opioid receptor. In one embodiment, the opioid antagonist is selected from the group consisting of alvimopan, norbinaltorphimine, nalmefene, naloxone, naltrexone, methylnaltrexone, and nalorphine, and pharmaceutically acceptable salts or prodrugs thereof. In another embodiment, the opioid antagonist is a partial opioid agonist, which is a weak agonist, such as pentacozine, buprenorphine, nalorphine, propiram, and lofexidine.

Moreover, several peptides and hormones regulate feeding behavior. For example, cholecystokinin and serotonin act to decrease appetite and food intake. Leptin, a hormone produced by fat cells, controls food intake and energy expenditure. In obese persons who are losing weight without medications, a decrease in weight is associated with a decrease in circulating levels of leptin, suggesting its role in weight homeostasis. Obese patients with high leptin levels are thought to have peripheral leptin resistance secondary to the down-regulation of leptin receptors. Non-limiting examples of useful compounds affecting feeding behavior include certain leptin-lipolysis stimulated receptors as disclosed by WO 01/21647 which is incorporated herein by reference; certain phosphodiesterase enzyme inhibitors as disclosed by WO 01/35970 which is incorporated herein by reference; certain compounds having nucleotide sequences of the mahogany gene as disclosed by WO 00/05373 which is incorporated herein by reference; and certain sapogenin compounds as disclosed by U.S. Pat. No. 4,680,289 which is incorporated herein by reference.

Other useful compounds include certain gamma peroxisome proliferator activated receptor (PPAR) agonists as disclosed by WO 01/30343 and U.S. Pat. No. 6,033,656 which are incorporated herein by reference and certain polypeptides such as fibroblast growth factor-10 polypeptides as disclosed by WO 01/18210 which is incorporated herein by reference.

Other useful compounds include GLP-1 and compounds with similar mechanisms of action, such as incretin mimetics, including specifically but without limitation exenatide (marketed as Byetta®), approved for the treatment of type 2 diabetes. Exenatide is disclosed in U.S. Pat. No. 5,424,286 which is incorporated herein by reference.

Dipeptidyl peptidase-4 (DPP-4) inhibitors, which control blood glucose values, are also useful in the practice of the present invention. On DPP-4 inhibitor is sitagliptin (marketed as Januvia®) and approved for the treatment of type 2 diabetes, and combinations of sitagliptin and other agents, including Janumet®, a combination of sitagliptin and metformin marketed in the United States. Various DPP-4 inhibitors and methods of use are disclosed in, for example, U.S. Pat. Nos. 6,303,661, 6,890,898, 6,699,871, 7,078,381 and 7,125,873.

Cannabinoid-1 (CB-1) receptor blockers or antagonists may be useful in the practice of the present invention, including compounds such as rimonabant, sold in countries in the European Union under the tradename Acomplia®. Various forms and formulations of rimonabant are disclosed in, for example, U.S. Pat. Nos. 5,462,960 and 5,624,941 and international patent applications WO 2007/090949 and WO 2008/026219.

Moreover, monoamine oxidase inhibitors that decrease energy intake or increase energy expenditure are useful with the practice of the present invention. Suitable, but non-limiting, examples of monoamine oxidase inhibitors include befloxatone, moclobemide, brofaromine, phenoxathine, esuprone, befol, toloxatone, pirlindol, amiflamine, sercloremine, bazinaprine, lazabemide, milacemide, caroxazone and other certain compounds as disclosed by WO 01/12176 which is incorporated herein by reference.

Certain compounds that increase lipid metabolism are also useful in the practice of the present invention. Such compounds include, but are not limited to, evodiamine compounds as disclosed by U.S. Pat. No. 6,214,831 which is incorporated herein by reference.

Nutrient partitioning agents and digestive inhibitors are another strategy in the treatment of obesity by interfering with the breakdown, digestion or absorption of dietary fat in the gastrointestinal tract. Gastric and pancreatic lipases aid in the digestion of dietary triglycerides by forming them into free fatty acids that are then absorbed in the small intestine. Inhibition of these enzymes leads to inhibition of the digestion of dietary triglycerides. Non-limiting examples include a lipase inhibitor, orlistat, such as XENICAL™ capsules ((S)-2-formylamino-4-methyl-pentanoic acid (S)-1-[[(2S,3S)-3-hexyl-4-oxo-2-oxetanyl]methyl]-dodecyl ester) commercially available from Roche Laboratories and certain benzoxazinone compounds as described by WO 00/40247 which is incorporated herein by reference.

Agents that increase energy expenditure are also referred to as thermogenic medications. Non-limiting examples of suitable thermogenic medications include xanthines, such as caffeine and theophylline, selective β-3-adrenergic agonists, for example certain compounds in U.S. Pat. No. 4,626,549 which is incorporated by reference herein, and α-2-adrenergic and growth hormones compounds as described in U.S. Pat. Nos. 4,937,267 and 5,120,713 which are incorporated by reference herein.

Generally, a total dosage of the above-described obesity control agents or medications, when used in combination with one or more peptides of the present invention can range from 0.1 to 3,000 mg/day, preferably from about 1 to 1,000 mg/day and more preferably from about 1 to 200 mg/day in single or 2-4 divided doses. The exact dose, however, is determined by the attending clinician and is dependent on such factors as the potency of the compound administered, the age, weight, condition and response of the patient.

Agents or drugs employed for increasing food intake and/or body weight include appetite stimulants such as megestrol acetate, adrenocorticoids such as prednisolone and dexamethasone, cyproheptidine, serotonergic drugs such as fenfluramine, neuropeptide Y, and androgen antagonists such as flutamide, nilutamide, and zanoterone.

### 4.0 Methods of Administration and Use

The method of administration and use varies depending upon the characteristic of specific peptides of the present invention, the disease, indication, condition or syndrome to be treated, and other factors known to those in the art. In general, any method of administration and use known in the art or hereafter developed may be employed with the peptides of the present invention. Without limiting the foregoing, the following methods of administration and use have specific application for the indicated indications.

4.1 Injection

Compositions including one or more peptides of the present invention may administered by any suitable means for therapy, including prophylactic therapy, of obesity and metabolic syndrome. In one aspect, the composition is formulated for subcutaneous injection, and a subcutaneous injection is given one or more times each day, preferably prior to a meal, more preferably between about one and about three hours prior to a mean. In another aspect, the composition is formulated as an injectable sustained release formulation. In one embodiment, a peptide of the present invention is formulated with a polyethylene glycol, such as polyethylene glycol 3350, and optionally one or more additional excipients and preservatives, including but not limited to excipients such as salts, polysorbate 80, sodium hydroxide or hydrochloric acid to adjust pH, and the like. In another embodiment a peptide of the present invention is formulated with a poly(ortho ester), which may be an auto-catalyzed poly(ortho ester) with any of a variable percentage of lactic acid in the polymeric backbone, and optionally one or more additional excipients. In one embodiment poly (D,L-lactide-co-glycolide) polymer (PLGA polymer) is employed, preferably a PLGA polymer with a hydrophilic end group, such as PLGA RG502H from Boehringer Ingelheim, Inc. (Ingelheim, Germany). Such formulations may be made, for example, by combining a peptide of the present invention in a suitable solvent, such as methanol, with a solution of PLGA in methylene chloride, and adding thereto a continuous phase solution of polyvinyl alcohol under suitable mixing conditions in a reactor. In general, any of a number of injectable and biodegradable polymers, which are preferably also adhesive polymers, may be employed in a sustained release injectable formulation. The teachings of U.S. Pat. Nos. 4,938,763, 6,432,438, and 6,673,767, and the biodegradable polymers and methods of formulation disclosed therein, are incorporated here by reference. The formulation may be such that an injection is required on a weekly, monthly or other periodic basis, depending on the concentration and amount of peptide, the biodegradation rate of the polymer, and other factors known to those of skill in the art.

4.2 Oral

Compositions including one or more peptides of the present invention may be administered orally in an individual dosage form such as a tablet or capsule. In one preferred aspect, the individual dosage form includes an enteric coating, and optionally one or more agents to increase uptake, decrease protease degradation, increase cellular permeability, and the like.

4.3 Other Administration

In one embodiment the composition is formulated for any of a variety of transdermal routes of administration, including buccal administration, nasal administration, inhalation administration and the like. Particularly preferred are embodiments wherein the composition is formulated for nasal administration, such as by means of a metered spray device delivering a volume of from about 20 to about 200 μL of an aqueous composition including any of a variety of other agents, including permeability enhancing agents.

### 5.0 Methods of Making

In general, the peptides of the present invention may be synthesized by solid-phase synthesis and purified according to methods known in the art. Any of a number of well-known procedures utilizing a variety of resins and reagents may be used to prepare the peptides of the present invention.

The cyclic peptides of the present invention may be readily synthesized by known conventional procedures for the formation of a peptide linkage between amino acids. Such conventional procedures include, for example, any solution phase procedure permitting a condensation between the free alpha amino group of an amino acid or residue thereof having its carboxyl group and other reactive groups protected and the free primary carboxyl group of another amino acid or residue thereof having its amino group or other reactive groups protected. In a preferred conventional procedure, the cyclic peptides of the present invention may be synthesized by solid-phase synthesis and purified according to methods known in the art. Any of a number of well-known procedures utilizing a variety of resins and reagents may be used to prepare the peptides of the present invention.

The process for synthesizing the cyclic peptides may be carried out by a procedure whereby each amino acid in the desired sequence is added one at a time in succession to another amino acid or residue thereof or by a procedure whereby peptide fragments with the desired amino acid sequence are first synthesized conventionally and then condensed to provide the desired peptide. The resulting peptide is then cyclized to yield a cyclic peptide of the invention.

Solid phase peptide synthesis methods are well known and practiced in the art. In such methods the synthesis of peptides of the invention can be carried out by sequentially incorporating the desired amino acid residues one at a time into the growing peptide chain according to the general principles of solid phase methods. These methods are disclosed in numerous references, including Merrifield, R. B., Solid phase synthesis (Nobel lecture). Angew Chem 24:799-810 (1985) and Barany et al., The Peptides, Analysis, Synthesis and Biology, Vol. 2, Gross, E. and Meienhofer, J., Eds. Academic Press 1-284 (1980).

In chemical syntheses of peptides, reactive side chain groups of the various amino acid residues are protected with suitable protecting groups, which prevent a chemical reaction from occurring at that site until the protecting group is removed. Also common is the protection of the alpha amino group of an amino acid residue or fragment while that entity reacts at the carboxyl group, followed by the selective removal of the alpha amino protecting group to allow a subsequent reaction to take place at that site. Specific protecting groups have been disclosed and are known in solid phase synthesis methods and solution phase synthesis methods.

Alpha amino groups may be protected by a suitable protecting group, including a urethane-type protecting group, such as benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz) and aliphatic urethane-type protecting groups, such as t-butyloxycarbonyl (Boc), diisopropylmethoxycarbonyl, isopropoxycarbonyl, and allyloxycarbonyl (Alloc). Fmoc is preferred for alpha amino protection.

Guanidino groups may be protected by a suitable protecting group, such as nitro, p-toluenesulfonyl (Tos), Z, pentamethylchromanesulfonyl (Pmc), adamantyloxycarbonyl, pentamethyldihydrobenzofuran-5-sulfonyl (Pbf) and Boc. Pbf is a preferred protecting group for Arg, but other protecting groups may be employed.

The peptides of the invention described herein were prepared using solid phase synthesis, such as by means of a Symphony Multiplex Peptide Synthesizer (Rainin Instrument Company) automated peptide synthesizer, using programming modules as provided by the manufacturer and following the protocols set forth in the manufacturer's manual.

Solid phase synthesis is commenced from the C-terminal end of the peptide by coupling a protected alpha amino acid to a suitable resin. Such starting material is prepared by attaching an alpha amino-protected amino acid by an ester linkage to a p-benzyloxybenzyl alcohol (Wang) resin, a 2-chlorotrityl chloride resin or an oxime resin, by an amide bond between an Fmoc-Linker, such as p-[(R, S)-α-[1-(9H-fluor-en-9-yl)-methoxyformamido]-2,4-dimethyloxybenzyl]-phenoxyacetic acid (Rink linker) to a benzhydrylamine (BHA) resin, or by other means well known in the art. Fmoc-Linker-BHA resin supports are commercially available and generally used when feasible. The resins are carried through repetitive cycles as necessary to add amino acids sequentially. The alpha amino Fmoc protecting groups are removed under basic conditions. Piperidine, piperazine, diethylamine, or morpholine (20-40% v/v) in N,N-dimethylformamide (DMF) may be used for this purpose.

Following removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. After the peptide is synthesized, if desired, the orthogonally protected side chain protecting groups may be removed using methods well known in the art for further derivatization of the peptide.

Typically, orthogonal protecting groups are used as appropriate. For example, the peptides of the invention contain multiple amino acids with an amino group-containing side chain. In one aspect, an Allyl-Alloc protection scheme is employed with the amino acids forming a lactam bridge through their side chains, and orthogonal protecting groups, cleavable under different reactive conditions, use for other amino acids with amino group-containing side chains. Thus, for example, Fmoc-Lys(Alloc)-OH, Fmoc-Orn(Alloc)-OH, Fmoc-Dap(Alloc)-OH, Fmoc-Dab(Alloc)-OH, Fmoc-Asp(OAII)-OH or Fmoc-Glu(OAII)-OH amino acids can be employed for the positions forming a lactam bridge upon cyclization, while other amino acids with amino group-containing side chains have a different and orthogonal protecting group, such as with Fmoc-Arg(Pbf)-OH, Fmoc-Lys(Pbf)-OH, Fmoc-Dab(Pbf)-OH or the like. Other protecting groups may be similarly employed; by way of example and not limitation, Mtt/OPp (4-methyltrityl/2-phenylisopropyl) can be employed with the side chains forming a lactam bridge upon cyclization, with orthogonal protecting groups being utilized for other positions that are not cleavable using conditions suitable for cleavage of Mtt/OPp.

Reactive groups in a peptide can be selectively modified, either during solid phase synthesis or after removal from the resin. For example, peptides can be modified to obtain N-terminus modifications, such as acetylation, while on resin, or may be removed from the resin by use of a cleaving reagent and then modified. Similarly, methods for modifying side chains of amino acids are well known to those skilled in the art of peptide synthesis. The choice of modifications made to reactive groups present on the peptide will be determined, in part, by the characteristics that are desired in the peptide.

In the peptides of the present invention, in one preferred embodiment the N-terminus group is modified by introduction of an N-acetyl group. In one aspect, a method is employed wherein, after removal of the protecting group at the N-terminal, the resin-bound peptide is reacted with acetic anhydride in dichloromethane in the presence of an organic base, such as diisopropylethylamine. Other methods of N-terminus acetylation are known in the art, including solution phase acetylation, and may be employed.

The peptide can, in one embodiment, be cyclized prior to cleavage from the peptide resin. For cyclization through reactive side chain moieties, the desired side chains are deprotected, and the peptide suspended in a suitable solvent and a cyclic coupling agent added. Suitable solvents include, for example DMF, dichloromethane (DCM) or 1-methyl-2-pyrrolidone (NMP). Suitable cyclic coupling reagents include, for example, 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP), 2-(7-aza-1H-benzotriazol-1-yl)-1,1,3,3-tetramethyluronium tetrafluoroborate (TATU), 2-(2-oxo-1 (2H)-pyridyl)-1,1,3,3-tetramethyluronium tetrafluoroborate (TPTU) or N,N′-dicyclohexylcarbodiimide/1-hydroxybenzotriazole (DCCl/HOBt). Coupling is conventionally initiated by use of a suitable base, such as N,N-diisopropylethylamine (DIPEA), sym-collidine or N-methylmorpholine (NMM).

The cyclized peptides can then be cleaved from solid phase, using any suitable reagent, such as ethylamine in DCM or various combinations of agents, such as trifluoroacetic acid (TFA), tri-isopropylsilane (TIS), dimethoxybenezene (DMB), water and the like. The resulting crude peptide is dried and remaining amino acid side chain protecting groups, if any, are cleaved using any suitable reagent, such as TFA in the presence of water, TIS, 2-mercaptopethane (ME), and/or 1,2-ethanedithiol (EDT). The final product is precipitated by adding cold ether and collected by filtration. Final purification is by reverse phase high performance liquid chromatography (RP-HPLC), using a suitable column, such as a C₁₈ column, or other methods of separation or purification, such as methods based on the size or charge of the peptide, can also be employed. Once purified, the peptide can be characterized by any number of methods, such as high performance liquid chromatograph (HPLC), amino acid analysis, mass spectrometry, and the like.

While synthesis has been described primarily with reference to solid phase Fmoc chemistry, it is to be understood that other chemistries and synthetic methods may be employed to make the cyclic peptides of the invention, such as by way of example and not limitation, methods employing Boc chemistry, solution chemistry, and other chemistries and synthetic methods.

### 6.0 Formulations

Depending on the desired route of administration, the formulation of a composition including one or more cyclic peptides of the present invention may be varied. Thus the formulation may be suitable for subcutaneous injection, or intravenous injection, for topical applications, for ocular applications, for nasal spray applications, for inhalation applications, for other transdermal applications and the like.

6.1 Salt Form of Cyclic Peptides of the Present Invention

The cyclic peptides of the present invention may be in the form of any pharmaceutically acceptable salt. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the cyclic peptide of the present invention is basic, acid addition salts may be prepared from pharmaceutically acceptable non-toxic acids, including inorganic and organic acids. Such acids include acetic, benzenesulfonic, benzoic, camphorsulfonic, carboxylic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Acid addition salts of the peptides of the present invention are prepared in a suitable solvent from the peptide and an excess of an acid, such as hydrochloric, hydrobromic, sulfuric, phosphoric, acetate, acetic, trifluoroacetic, citric, tartaric, maleic, succinic or methanesulfonic acid. The acetate, ammonium acetate and trifluoroacetic acid salt forms are especially useful. Where the peptides of the present invention include an acidic moiety, suitable pharmaceutically acceptable salts may include alkali metal salts, such as sodium or potassium salts, or alkaline earth metal salts, such as calcium or magnesium salts.

6.2 Pharmaceutical Compositions

The invention provides a pharmaceutical composition that includes a cyclic peptide of the present invention and a pharmaceutically acceptable carrier. The carrier may be a liquid formulation, and is preferably a buffered, isotonic, aqueous solution. Pharmaceutically acceptable carriers also include excipients, such as diluents, carriers and the like, and additives, such as stabilizing agents, preservatives, solubilizing agents, buffers and the like, as hereafter described.

The cyclic peptide compositions of the present invention may be formulated or compounded into pharmaceutical compositions that include at least one cyclic peptide of the present invention together with one or more pharmaceutically acceptable carriers, including excipients, such as diluents, carriers and the like, and additives, such as stabilizing agents, preservatives, solubilizing agents, buffers and the like, as may be desired. Formulation excipients may include polyvinylpyrrolidone, gelatin, hydroxy cellulose, acacia, polyethylene glycol, manniton, sodium chloride and sodium citrate. For injection or other liquid administration formulations, water containing at least one or more buffering constituents is preferred, and stabilizing agents, preservatives and solubilizing agents may also be employed. For solid administration formulations, any of a variety of thickening, filler, bulking and carrier additives may be employed, such as starches, sugars, fatty acids and the like. For topical administration formulations, any of a variety of creams, ointments, gels, lotions and the like may be employed. For most pharmaceutical formulations, non-active ingredients will constitute the greater part, by weight or volume, of the preparation. For pharmaceutical formulations, it is also contemplated that any of a variety of measured-release, slow-release or sustained-release formulations and additives may be employed, so that the dosage may be formulated so as to effect delivery of a peptide of the present invention over a period of time.

In general, the actual quantity of cyclic peptides of the present invention administered to a patient will vary between fairly wide ranges depending on the mode of administration, the formulation used, and the response desired.

In practical use, the cyclic peptides of the invention can be combined as the active ingredient in an admixture with a pharmaceutical carrier according to conventional pharmaceutical compounding techniques. The carrier may take a wide variety of forms depending on the form of preparation desired for administration, for example, oral, parenteral (including intravenous), urethral, vaginal, nasal, buccal, sublingual, or the like. In preparing the compositions for oral dosage form, any of the usual pharmaceutical media may be employed, such as, for example, water, glycols, oils, alcohols, flavoring agents, preservatives, coloring agents and the like in the case of oral liquid preparations such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets.

Because of their ease of administration, tablets and capsules represent an advantageous oral dosage unit form. If desired, tablets may be coated by standard aqueous or nonaqueous techniques. The amount of active peptide in such therapeutically useful compositions is such that an effective dosage will be obtained. In another advantageous dosage unit form, sublingual constructs may be employed, such as sheets, wafers, tablets or the like.

The tablets, pills, capsules, and the like may also contain a binder such as gum tragacanth, acacia, corn starch or gelatin; excipients such as dicalcium phosphate; a disintegrating agent such as corn starch, potato starch or alginic acid; a lubricant such as magnesium stearate; and a sweetening agent such as sucrose, lactose or saccharin. When a dosage unit form is a capsule, it may contain, in addition to materials of the above type, a liquid carrier such as fatty oil.

Various other materials may be utilized as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Cyclic peptides may also be administered parenterally. Solutions or suspensions of these active peptides can be prepared in water suitably mixed with a surfactant such as hydroxy-propylcellulose. Dispersions can also be prepared in glycerol, liquid polyethylene glycols and mixtures thereof in oils. These preparations may optionally contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that it may be administered by syringe. The form must be stable under the conditions of manufacture and storage and must be preserved against the contaminating action of microorganisms such as bacteria and fungi. The carrier can be a solvent or dispersion medium containing, for example, water, ethanol, a polyol, for example glycerol, propylene glycol or liquid polyethylene glycol, suitable mixtures thereof, and vegetable oils.

The cyclic peptides of the present invention may be therapeutically applied by means of nasal administration. By “nasal administration” is meant any form of intranasal administration of any of the cyclic peptides of the present invention. The peptides may be in an aqueous solution, such as a solution including saline, citrate or other common excipients or preservatives. The peptides may also be in a dry or powder formulation.

The cyclic peptides of the present invention may be formulated with any of a variety of agents that increase effective nasal absorption of drugs, including peptide drugs. These agents should increase nasal absorption without unacceptable damage to the mucosal membrane. U.S. Pat. Nos. 5,693,608, 5,977,070 and 5,908,825, among others, teach a number of pharmaceutical compositions that may be employed, including absorption enhancers, and the teachings of each of the foregoing, and all references and patents cited therein, are incorporated by reference.

If in an aqueous solution, the cyclic peptides may be appropriately buffered by means of saline, acetate, phosphate, citrate, acetate or other buffering agents, which may be at any physiologically acceptable pH, generally from about pH 4 to about pH 7. A combination of buffering agents may also be employed, such as phosphate buffered saline, a saline and acetate buffer, and the like. In the case of saline, a 0.9% saline solution may be employed. In the case of acetate, phosphate, citrate, and the like, a 50 mM solution may be employed. In addition to buffering agents, a suitable preservative may be employed, to prevent or limit bacteria and other microbial growth. One such preservative that may be employed is 0.05% benzalkonium chloride.

In an alternative embodiment, cyclic peptides of the present invention may be administered directly into the lung. Intrapulmonary administration may be performed by means of a metered dose inhaler, a device allowing self-administration of a metered bolus of a peptide of the present invention when actuated by a patient during inspiration. In one aspect of this embodiment, the cyclic peptide may be in a dried and particulate form, for example particles between about 0.5 and 6.0 μm, such that the particles have sufficient mass to settle on the lung surface, and not be exhaled, but are small enough that they are not deposited on surfaces of the air passages prior to reaching the lung. Any of a variety of different techniques may be used to make dry powder microparticles, including but not limited to micro-milling, spray drying and a quick freeze aerosol followed by lyophilization. With micro-particles, the peptides may be deposited to the deep lung, thereby providing quick and efficient absorption into the bloodstream. Further, with such approach penetration enhancers are not required, as is sometimes the case in transdermal, nasal or oral mucosal delivery routes. Any of a variety of inhalers can be employed, including propellant-based aerosols, nebulizers, single dose dry powder inhalers and multidose dry powder inhalers. Common devices in current use include metered dose inhalers, which are used to deliver medications for the treatment of asthma, chronic obstructive pulmonary disease and the like. Preferred devices include dry powder inhalers, designed to form a cloud or aerosol of fine powder with a particle size that is always less than about 6.0 μm.

Microparticle size, including mean size distribution, may be controlled by means of the method of making. For micro-milling, the size of the milling head, speed of the rotor, time of processing and the like control the microparticle size. For spray drying, the nozzle size, flow rate, dryer heat and the like control the microparticle size. For making by means of quick freeze aerosol followed by lyophilization, the nozzle size, flow rate, concentration of aerosoled solution and the like control the microparticle size. These parameters and others may be employed to control the microparticle size.

The cyclic peptides of the present invention may be therapeutically administered by means of an injection of a sustained release formulation. In one embodiment, a cyclic peptide of the present invention is formulated for a deep intramuscular injection, such as in the gluteal or deltoid muscle, of a formulation with a polyethylene glycol, such as polyethylene glycol 3350, and optionally one or more additional excipients and preservatives, including but not limited to excipients such as salts, polysorbate 80, sodium hydroxide or hydrochloric acid to adjust pH, and the like. In another embodiment a cyclic peptide of the present invention is formulated with a poly(ortho ester), which may be an auto-catalyzed poly(ortho ester) with any of a variable percentage of lactic acid in the polymeric backbone, and optionally one or more additional excipients. In one embodiment poly (D,L-lactide-co-glycolide) polymer is employed. In general, any of a number of injectable and bioerodible polymers, which are preferably also adhesive polymers, may be employed in a sustained release injectable formulation. Alternatively other sustained release formulations may be employed, including formulations permitting subcutaneous injection, which other formulations may include one or more of nano/microspheres (such as compositions including PLGA polymers), liposomes, emulsions (such as water-in-oil emulsions), gels, insoluble salts or suspensions in oil. The formulation may be such that an injection is required on a daily, weekly, monthly or other periodic basis, depending on the concentration and amount of cyclic peptide, the sustained release rate of the materials employed, and other factors known to those of skill in the art.

6.3 Oral Formulations of Peptides of the Present Invention

In one aspect, the peptides of the present invention are formulated for oral delivery. The peptide is preferably formulated and made such that it is encased in an enteric protectant, more preferably such that it is not released until the tablet or capsule has transited the stomach, and optionally has further transited a portion of the small intestine. In the context of this application it will be understood that the term enteric coating or material refers to a coating or material that will pass through the stomach essentially intact but will rapidly disintegrate in the small intestine to release the active drug substance. One enteric coating solution that may be used includes cellulose acetate phthalate, and optionally other ingredients such as ammonium hydroxide, triacetin, ethyl alcohol, methylene blue, and purified water. Cellulose acetate phthalate is a polymer that has been used in the pharmaceutical industry for enterically coating individual dosage forms such as tablets and capsules, and is not soluble in water at a pH of less than about 5.8. Enteric coatings including cellulose acetate phthalate provide protection against the acidic environment of the stomach, but begin to dissolve in environment of the duodenum (pH of about 6-6.5), and are completely dissolved by the time the dosage form reaches the ileum (pH of about 7-8). In addition to cellulose acetate phthalate, other enteric coating materials are known and may be used with peptides of the present invention, including without limitation hydroxypropylmethylethylcellulose succinate, hydroxypropylmethylcellulose phthalate, polyvinyl acetate phthalate, and methacrylic acid-methyl methacrylate copolymer. The enteric coating employed promotes dissolution of the dosage form primarily at a site outside the stomach, and may be selected such that the enteric coating dissolves at a pH of approximately at least 6.0, more preferable at a pH of from about 6.0 to about 8.0. In one preferred aspect, the enteric coating dissolves and breaks down in the proximity of the ileum.

Any of a variety of permeation enhancers may be employed, to increase uptake in the intestines upon dissolution of the enteric coating. In one aspect, permeation enhancers increase either paracellular or transcellular transport systems. An increase in paracellular transport can be achieved by opening the tight junctions of the cells; an increase in transcellular transport can be achieved by increasing the fluidity of the cell membrane. Representative, non-limiting examples of such permeation enhancers include calcium chelators, bile salts (such as sodium cholate), and fatty acids. The peptides of the present invention may be in an enteric-coated individual dosage form that includes a fatty acid, such as for example oleate, palmitate, stearate, sodium caprate, or conjugated linoleic acid, in an enteric-coated capsule, to increase paracellular transport.

In one aspect, the individual dosage form, such as a tablet or capsule, optionally further includes common pharmaceutical binders such as povidone, diluents, glidants, fillers such as microcrystalline cellulose, lubricants such as magnesium stearate, disintegrants such as croscarmellose sodium, preservatives, colorants and the like in their usual known sizes and amounts. In some embodiments, peptides or polypeptides that act as substrates for intestinal proteases are further added.

6.4 Routes of Administration

If a composition including one or more peptides of the present invention is administered by injection, the injection may be intravenous, subcutaneous, intramuscular, intraperitoneal or other means known in the art. The peptides of the present invention may be formulated by any means known in the art, including but not limited to formulation as tablets, capsules, caplets, suspensions, powders, lyophilized preparations, suppositories, ocular drops, skin patches, oral soluble formulations, sprays, aerosols and the like, and may be mixed and formulated with buffers, binders, excipients, stabilizers, anti-oxidants and other agents known in the art. In general, any route of administration by which the peptides of invention are introduced across an epidermal layer of cells may be employed. Administration means may thus include administration through mucous membranes, buccal administration, oral administration, dermal administration, inhalation administration, nasal administration, urethral administration, vaginal administration, and the like.

6.5 Therapeutically Effective Amount

In general, the actual quantity of cyclic peptide of the present invention administered to a patient will vary between fairly wide ranges depending upon the mode of administration, the formulation used, and the response desired. The dosage for treatment is administration, by any of the foregoing means or any other means known in the art, of an amount sufficient to bring about the desired therapeutic effect. Thus a therapeutically effective amount includes an amount of a peptide or pharmaceutical composition of the present invention that is sufficient to therapeutically alleviate sexual dysfunction in a patient, or to prevent or delay onset or recurrence of the sexual dysfunction.

In general, the cyclic peptides of the present invention are highly active. For example, the cyclic peptide can be administered at about 0.1, 0.5, 1, 5, 50, 100, 500, 1000 or 5000 μg/kg body weight, depending on the specific peptide selected, the desired therapeutic response, the route of administration, the formulation and other factors known to those of skill in the art.

### 7.0 Tests and Assays Employed in Evaluation of the Peptides of the Present Invention

The melanocortin receptor-specific peptides of the present invention of this invention may be tested by a variety of assay systems and animal models to determine binding, functional status and efficacy.

7.1 Competitive Inhibition Assay Using [I¹²⁵]-NDP-α-MSH

Competitive inhibition binding assays were performed using membrane homogenates prepared from HEK-293 cells that express recombinant hMC1-R, hMC3-R, hMC4-R, or hMC5-R. In the examples that follow, all MC1-R, MC3-R, MC4-R and MC5-R values are for human recombinant receptors. Assays were performed in 96 well GF/B Millipore multiscreen filtration plates (MAFB NOB10) pre-coated with 0.5% bovine serum albumin (Fraction V). Membrane homogenates were incubated with 0.1 nM [1¹²⁵]-NDP-α-MSH (Perkin Elmer) and increasing concentrations of test peptides of the present invention in buffer containing 25 mM HEPES buffer (pH 7.5) with 100 mM NaCl, 2 mM CaCl₂, 2 mM MgCl₂, 0.3 mM 1,10-phenanthroline, and 0.1% bovine serum albumin. After incubation for 90 minutes at 37° C., the assay mixture was filtered and the membranes washed with 1 mL of cold 1×PBS wash buffer per well. 35 μL of scintillation cocktail was added to each well and the plates counted on a Microbeta counter for 1 minute per well. Non-specific binding was measured by inhibition of binding of [I¹²⁵]-NDP-α-MSH in the presence of 1 μM NDP-α-MSH. Maximal specific binding (100%) was defined as the difference in radioactivity (cpm) bound to cell membranes in the absence and presence of 1 μM NDP-α-MSH. Radioactivity (cpm) obtained in the presence of test compounds was normalized with respect to 100% specific binding to determine the percent inhibition of [I¹²⁵]-NDP-α-MSH binding. Each assay was conducted in duplicate and actual mean values are described, with results less than 0% reported as 0%. Ki values for test peptides of the present invention were determined using Graph-Pad Prism® curve-fitting software.

7.2 Competitive Binding Assay Using Eu-NDP-α-MSH

Alternatively, a competitive inhibition binding assay was performed employing Eu-NDP-α-MSH (PerkinElmer Life Sciences catalog No. AD0225) with determination by time-resolved fluorometry (TRF) of the lanthanide chelate. In comparison studies with [I¹²⁵]-NDP-α-MSH, the same values, within experimental error ranges, were obtained for percent inhibition and Ki. Typically competition experiments to determine Ki values were conducted by incubating membrane homogenates prepared from HEK-293 cells that express recombinant hMC4-R with 9 different concentrations of test compounds of interest and 1 nM of Eu-NDP-α-MSH in a solution containing 25 mM HEPES buffer with 100 mM NaCl, 2 mM CaCl₂, 2 mM MgCl₂, 0.1% BSA, and 0.3 mM 1,10-phenanthroline. After incubation for 90 minutes at 37° C., the reaction was stopped by filtration over AcroWell 96-well filter plates (Pall Life Sciences). The filter plates were washed 4 times with 200 μL of ice-cold phosphate-buffered saline. DELFIA Enhancement solution (PerkinElmer Life Sciences) was added to each well. The plates were incubated on a shaker for 15 minutes and read at 340 nm excitation and 615 nm emission wavelengths. Each assay was conducted in duplicate and mean values were utilized. Ki values were determined by curve-fitting with Graph-Pad Prism® software using a one-site fixed-slope competition binding model.

7.3 Assay for Agonist Activity

Accumulation of intracellular cAMP was examined as a measure of the ability of the peptides of the present invention to elicit a functional response in HEK-293 cells that express MC4-R. Confluent HEK-293 cells that express recombinant hMC4-R were detached from culture plates by incubation in enzyme-free cell dissociation buffer. Dispersed cells were suspended in Earle's Balanced Salt Solution containing 10 mM HEPES (pH 7.5), 1 mM MgCl2, 1 mM glutamine, 0.5% albumin and 0.3 mM 3-isobutyl-1-methyl-xanthine (IBMX), a phosphodiesterase inhibitor. The cells were plated in 96-well plates at a density of 0.5×105 cells per well and pre-incubated for 10 minutes. Cells were exposed for 15 minutes at 37° C. to peptides of the present invention dissolved in DMSO (final DMSO concentration of 1%) at a concentration range of 0.05-5000 nM in a total assay volume of 200 μL. NDP-α-MSH was used as the reference agonist. cAMP levels were determined by an HTRF® cAMP cell-based assay system from Cisbio Bioassays utilizing cryptate-labeled anti-cAMP and d2-labeled cAMP, with plates read on a Perkin-Elmer Victor plate reader at 665 and 620 nM. Data analysis was performed by nonlinear regression analysis with Graph-Pad Prism® software. The maximum efficacies of the test peptides of the present invention were compared to that achieved by the reference melanocortin agonist NDP-α-MSH.

7.4 Food Intake and Body Weight Change

Change in food intake and body weight was evaluated for selected peptides administered by intravenous (IV) or subcutaneous injection routes. Male Sprague-Dawley rats were obtained from Hilltop Lab Animals, Inc. (Scottsdale, Pa.) or other vendors. Animals were individually housed in conventional polystyrene hanging cages and maintained on a controlled 12 hour on/off light cycle. Water and pelleted food was provided ad libitum. The rats were dosed IV with vehicle or selected peptides (0.3 to 1.0 mg/kg), or dosed subcutaneously with vehicle or selected peptides (doses up to 30 mg/kg). The changes in body weight and food intake for the 24 hour period after dosing was determined. The changes in body weight and food intake for the 48 hour and 72 hour periods after dosing can also be measured to determine reversal of changes in body weight and food intake effects back to baseline levels.

7.5 Induction of Penile Erection

The ability of peptides of the present invention to induce penile erection (PE) in male rats were evaluated with selected peptides. Male Sprague-Dawley rats weighing 250-300 g were kept on a 12 hour on/off light cycle with food and water ad libitum. All behavioral studies were performed between 9 a.m. and 4 p.m. Groups of 6-8 rats were administered peptides at a variety of doses via an IV route. Immediately after treatment, rats were placed into individual polystyrene cages (27 cm long, 16 cm wide, and 25 cm high) for behavioral observation, typically by remote video monitoring. Rats are observed for one hour, and the number of yawns, grooming bouts and PEs are recorded in 10-minute bins.

### 8.0 Peptides of the Invention

The peptides encompassed within formula (I) contain one or more asymmetric elements such as stereogenic centers, stereogenic axes and the like, so that the peptides encompassed within formula (I) can exist in different stereoisomeric forms. For both specific and generically described peptides, including the peptides encompassed within formula (I), all forms of isomers at all chiral or other isomeric centers, including enantiomers and diastereomers, are intended to be covered herein. The peptides of the invention each include multiple chiral centers, and may be used as a racemic mixture or an enantiomerically enriched mixture, in addition to use of the peptides of the invention in enantiopure preparations. Typically, the peptides of the invention will be synthesized with the use of chirally pure reagents, such as specified L- or D-amino acids, using reagents, conditions and methods such that enantiomeric purity is maintained, but it is possible and contemplated that racemic mixtures may be made. Such racemic mixtures may optionally be separated using well-known techniques and an individual enantiomer may be used alone. In cases and under specific conditions of temperature, solvents and pH wherein peptides may exist in tautomeric forms, each tautomeric form is contemplated as being included within this invention whether existing in equilibrium or predominantly in one form. Thus a single enantiomer of a peptide of formula (I), which is an optically active form, can be obtained by asymmetric synthesis, synthesis from optically pure precursors, or by resolution of the racemates.

The peptides of formulas (II) and (III) are specific stereoisomeric forms of the peptides of formula (I), but the invention should not be construed as being limited to the stereoisomeric forms encompassed by formulas (II) and (III).

The invention is further intended to include prodrugs of the present peptides, which on administration undergo chemical conversion by metabolic processes before becoming active pharmacological peptides. In general, such prodrugs will be functional derivatives of the present peptides, which are readily convertible in vivo into a peptide of formula (I). Prodrugs are any covalently bonded compounds, which release the active parent peptide drug of formula (I) in vivo. Conventional procedures for the selection and preparation of suitable prodrug derivatives are described, for example, in “Design of Prodrugs”, ed. H. Bundgaard, Elsevier, 1985. Typical examples of prodrugs have biologically labile protecting groups on a functional moiety, such as for example by esterification of hydroxyl, carboxyl or amino functions. Thus by way of example and not limitation, a prodrug includes peptides of formula (I) wherein an ester prodrug form is employed. Broadly speaking, prodrugs include compounds that can be oxidized, reduced, aminated, deaminated, hydroxylated, dehydroxylated, hydrolyzed, dehydrolyzed, alkylated, dealkylated, acylated, deacylated, phosphorylated or dephosphorylated to produce an active parent peptide drug of formula (I) in vivo.

The subject invention also includes peptides which are identical to those recited in formula (I), but for the fact that one or more atoms depicted in formula (I) are replaced by an atom having an atomic mass or mass number different from the atomic mass or mass number usually found in nature. Examples of isotopes that can be incorporated into compounds of the invention include isotopes of hydrogen, carbon, nitrogen and oxygen, such as ²H, ³H, ¹¹C, ¹³C, ¹⁴C, ¹⁵N, ¹⁸O and ¹⁷O, respectively. Peptides of the present invention and pharmaceutically acceptable salts or solvates of said compounds which contain the aforementioned isotopes and/or other isotopes of other atoms are within the scope of this invention. Certain isotopically-labeled compounds of the present invention, for example those into which radioactive isotopes such as ³H and ¹⁴C are incorporated, may have use in a variety of assays, such as in drug and/or substrate tissue distribution assays. Substitution with heavier isotopes, such as substitution of one or more hydrogen atoms with deuterium (²H), can provide pharmacological advantages in some instances, including increased metabolic stability. Isotopically labeled peptides of formula (I) can generally be prepared by substituting an isotopically labeled reagent for a non-isotopically labeled reagent.

8.1 Specific Peptides

Peptides of the following structures were synthesized by the general methods described above, and except where indicated MC4-R Ki values for each peptide were determined in competitive binding assays using Eu-NDP-α-MSH as described in 7.2 above.

| | | | MC4-R |
|---|---|---|---|
| No. | Structure | Amino Acid Sequence | Ki (nM) |
| 1 | 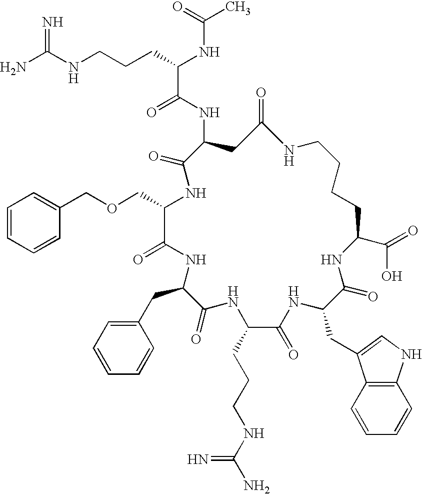 | Ac-Arg-cyclo(Asp-Ser(Bzl)-D- Phe-Arg-Trp-Lys)-OH (SEQ ID NO: 6) | 12 |
| 2 | 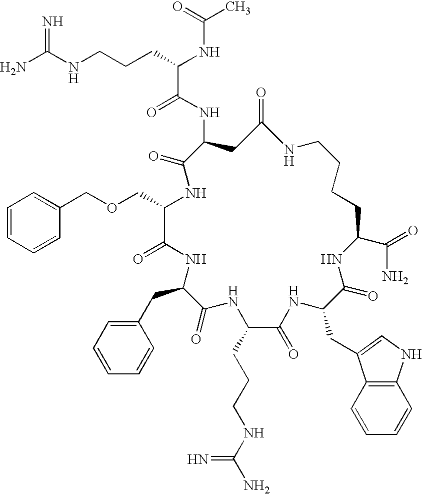 | Ac-Arg-cyclo(Asp-Ser(Bzl)-D- Phe-Arg-Trp-Lys)-NH₂ (SEQ ID NO: 7) | 1 |
| 3 | 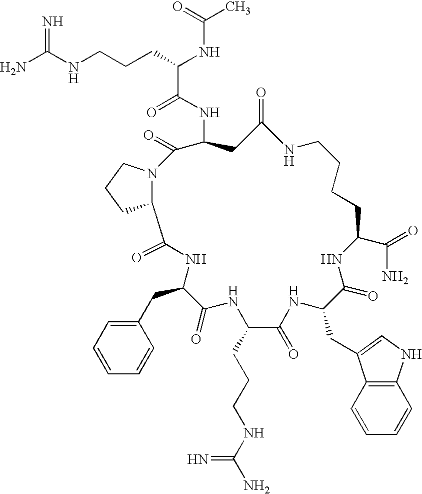 | Ac-Arg-cyclo(Asp-Pro-D-Phe- Arg-Trp-Lys)-NH₂ (SEQ ID NO: 8) | 11 |
| 4 | 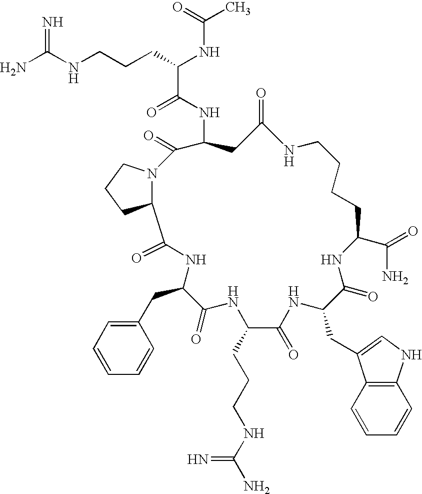 | Ac-Arg-cyclo(Asp-D-Pro-D- Phe-Arg-Trp-Lys)-NH₂ (SEQ ID NO: 11) | ND |
| 5 | 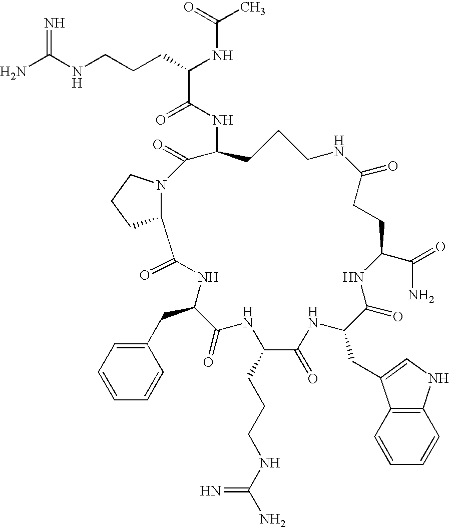 | Ac-Arg-cyclo(Orn-Pro-D-Phe- Arg-Trp-Glu)-NH₂ (SEQ ID NO: 9) | 13 |
| 6 | 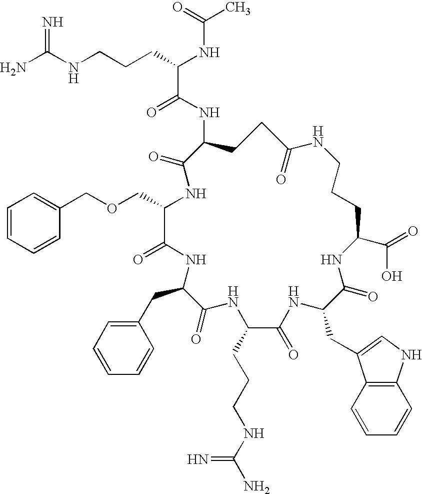 | Ac-Arg-cyclo(Glu-Ser(Bzl)-D- Phe-Arg-Trp-Orn)-OH (SEQ ID NO: 4) | 13 |
| 7 | 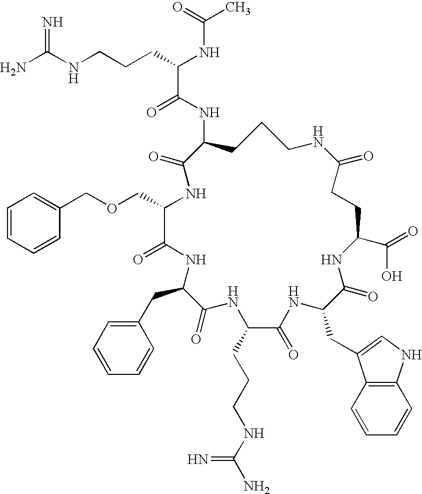 | Ac-Arg-cyclo(Orn-Ser(Bzl)-D- Phe-Arg-Trp-Glu)-OH (1SEQ ID NO: 5) | 9 |
| 8 | 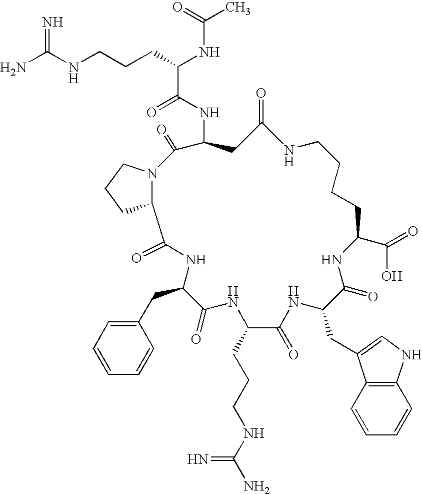 | Ac-Arg-cyclo(Asp-Pro-D-Phe- Arg-trp-Lys)-OH (SEQ ID NO: 10) | 116 |

8.2 Experiments with Compound 1

The cyclic peptide of Compound 1, with the formula Ac-Arg-cyclo(Asp-Ser(Bzl)-D-Phe-Arg-Trp-Lys)-OH, was made by solid phase peptide synthesis. The starting resin was 4-methoxybenzhydryl bromide resin, or, in other synthetic runs, Fmoc-Lys(Mtt)-p-alkoxybenzylalcohol resin. Protected amino acid derivatives were added sequentially, with Fmoc deprotection and peptide elongation employing standard Fmoc peptide chemistry. Generally, Fmoc was removed by mixing a solution of 20% piperidine in DMF for twenty minutes, and peptide coupling effected by mixing the Fmoc-amino acid (4 eq.), TBTU (4 eq.) and N-ethyl-N-(1-methylethyl)-2-propanamine (DIEA) (8 eq.) with the resin in DMF for thirty to sixty minutes. Where the starting resin was 4-methoxybenzhydryl bromide resin, the protected amino acid derivatives added sequentially were Fmol-Lys(Alloc)-OH, Fmoc-Trp(Boc)-OH, Fmoc-Arg(pbf)-OH, Fmoc-D-Phe-OH, Fmoc-Ser(Bzl)-OH, Fmoc-Asp(OAII)-OH, and Fmoc-Arg(Pbf)-OH. Where the starting resin was Fmoc-Lys(Mtt)-p-alkoxybenzylalcohol resin, the protected amino acid derivatives added sequentially were Fmoc-Trp(Boc)-OH, Fmoc-Arg(pbf)-OH, Fmoc-D-Phe-OH, Fmoc-Ser(Bzl)-OH, Fmoc-Asp(OPp)-OH, and Fmoc-Arg(Pbf)-OH. The Allyl and Alloc groups were simultaneously removed by treating the peptide resin with Pd(Ph₃P)₄ (0.2 eq.) and phenylsilane (20 eq.) in DCM under N₂ bubbling for thirty minutes, with the deprotection process repeated up to three additional times. The Mtt and OPp groups were removed by twice treating the peptide resin with 2% TFA and 2% triisopropylsilane (TIS) in DCM for twenty minutes. After Allyl/Alloc or Mtt/OPp deprotection, as appropriate, the lactam ring was formed on resin using TBTU (2 eq.) and DIEA (4 eq.) in DMF for one hour, or alternatively using PyBOP (4 eq.) and DIEA (8 eq.) in DMF over night, with coupling completion determined by Kaiser ninhydrin assay and, as needed, repeat couple. The peptide acid was cleaved by suspension in a cocktail of TFA/TIS/H₂O (95:2.5:2.5) for two hours followed by concentration and washing with cold diethyl ether. As necessary, any remaining amino acid side chain protecting groups, if any, are cleaved using any suitable reagent, such as TFA in the presence of water and EDT. Acyetylation is typically performed prior to cleavage of the peptide from resin. Following cleaving and washing, the peptide is purified, such as by RP-HPLC.

The cyclic peptide of Compound 1 was prepared as the acetate (AcOH) and trifluoroacetic acid (TFA) salt forms, and was also made in a form without salts. The cyclic peptide of Compound 1 has the molecular formula C₅₄H₇₃N₁₅O₁₁, and has a calculated molecular weight of 1108.25. The molecular weight of the cyclic peptide of Compound 1 as the acetate salt form was 1228.35, and as the TFA salt form was 1336.29.

The cyclic peptide of Compound 1 was evaluated for binding against MC1-R, MC3-R and MC4-R in competitive studies using Eu-NDP-α-MSH, and was found to be a highly selective for MC4-R, with a Ki value of 12 nM at MC4-R (average of nine studies), a Ki value of 1119 nM for MC1-R (average of three studies) and a Ki value of 1254 nM for MC3-R (average of two studies). In functional studies, the cyclic peptide of Compound 1 was determined to be a partial agonist, with intrinsic activity of 38% at MC4-R where NDP-α-MSH is 100%, and with an EC₅₀ of 6 nM (average of ten studies).

In rat feeding studies, using bremelanotide (a non-specific MC4-R agonist of the formula Ac-Nle-cyclo(Asp-His-D-Phe-Arg-Trp-Lys)-OH)) as a positive control, the cyclic peptide of Compound 1 was found to reduce food intake and decrease the rate of change in body weight. Using the methods as described above, groups of 8 rats each rats received 1 mg/kg bremelanotide, 0.3 mg/kg of the cyclic peptide of Compound 1, 1 mg/kg of the cyclic peptide of Compound 1 or 3.2% mannitol control. For the 0-2, 0-4, and 0-20 hour periods, the decrease in food consumption in rats receiving 1 mg/kg of the cyclic peptide of Compound 1 was statistically significant compared to control, while for the 0-4 hour period the decrease in food consumption in rats receiving 0.3 mg/kg of the cyclic peptide of Compound 1 was statistically significant compared to control. The 0-20 hour percent change in body weight was also statistically significant compared to control for the group receiving 1 mg/kg of the cyclic peptide of Compound 1.

In rat penile erection studies, again using bremelanotide as a positive control, the cyclic peptide of Compound 1 was not found to result in a statistically significant increase in observed spontaneous erections when administered IV. Vehicle alone resulted in an average of 0.5±0.189 spontaneous erections per rat in one hour (n=8), the cyclic peptide of Compound 1 resulted in an average of 1.5±0.378 spontaneous erections per rat in one hour (n=8), and bremelanotide resulted in a statistically significant average of 4.875±0.990 spontaneous erections per rat in one hour (n=8). Both the cyclic peptide of Compound 1 and bremelanotide were administered at a dose of 1 mg/kg.

8.3 Experiments with Compound 3

The cyclic peptide of Compound 3, with the formula Ac-Arg-cyclo(Asp-Pro-D-Phe-Arg-Trp-Lys)-NH₂, was made by the methods as described in 8.2 above for Compound 1, except that Fmoc-Rink resin (4-(2′,4′-dimethoxyphenyl-Fmoc-aminomethyl)-phenoxypolystyrene resin) was employed, Fmoc-Pro was substituted for Fmoc-Ser(Bzl), and cleavage from solid phase was by suspending the resin in a cocktail consisting of TFA/TIS/H₂O/DMB (90:2.5:2.5:5.0) for two hours. The cyclic peptide of Compound 3 was prepared as the TFA salt form. The cyclic peptide of Compound 3 has the molecular formula C₄₉H₇₀N₁₆O₉, and has a calculated molecular weight of 1027.18. The molecular weight of the cyclic peptide of Compound 3 as the TFA salt form was 1255.22.

The cyclic peptide of Compound 3 was evaluated for binding against MC1-R, MC3-R and MC4-R in competitive studies using NDP-α-MSH, and was found to be a highly selective for MC4-R, with a Ki value of 11 nM at MC4-R (average of two studies) and a Ki value of 125 nM for MC3-R (average of two studies). In functional studies, the cyclic peptide of Compound 1 was determined to be a partial agonist, with intrinsic activity of 47% at MC4-R where NDP-α-MSH is 100%, and with an EC₅₀ of 10 nM (average of three studies).

In rat feeding studies, using bremelanotide as a positive control, the cyclic peptide of Compound 3 was found to reduce food intake and decrease the rate of change in body weight. Using the methods as described above, groups of 8 rats each rats received 1 mg/kg bremelanotide, 0.3 mg/kg of the cyclic peptide of Compound 3, 1 mg/kg of the cyclic peptide of Compound 3 or 3.2% mannitol control. For the 0-2, 0-4, and 0-20 hour periods, the decrease in food consumption in rats receiving either 0.3 or 1 mg/kg of the cyclic peptide of Compound 3 was statistically significant compared to control. The 0-20 hour percent change in body weight was also statistically significant compared to control for the group receiving 1 mg/kg of the cyclic peptide of Compound 3.

In rat penile erection studies, again using bremelanotide as a positive control, the cyclic peptide of Compound 3 was not found to result in a statistically significant increase in observed spontaneous erections when administered by IV means. Vehicle alone resulted in an average of 0.714±0.286 spontaneous erections per rat in one hour (n=7), the cyclic peptide of Compound 3 at a dose of 0.3 mg/kg resulted in an average of 1.143±0.553 spontaneous erections per rat in one hour and at a dose of 1.0 mg/kg resulted in an average of 1.286±0.421 spontaneous erections per rat in one hour (both at n=7), and bremelanotide resulted in a statistically significant average of 3.714±0.680 spontaneous erections per rat in one hour (n=7).

8.4 Experiments with Compound 7

The cyclic peptide of Compound 7, with the formula Ac-Arg-cyclo(Orn-Ser(Bzl)-D-Phe-Arg-Trp-Glu)-OH, was made by the methods as described in 8.2 above for Compound 1, using 4-methoxybenzhydryl bromide resin as the starting resin, and with Fmoc-Glu(OAII)-OH substituted for Fmoc-Lys(Alloc) and Fmoc-Orn(Alloc) used rather than Fmoc-Asp(OAII)-OH. The cyclic peptide of Compound 7 was prepared as the TFA salt form. The cyclic peptide of Compound 7 has the molecular formula C₅₄H₇₃N₁₅O₁₁, and has a calculated molecular weight of 1108.25. The molecular weight of the cyclic peptide of Compound 7 as the TFA salt form was 1336.29.

The cyclic peptide of Compound 7 was evaluated for binding against MC1-R, MC3-R and MC4-R in competitive studies using NDP-α-MSH, and was found to be a highly selective for MC4-R, with a Ki value of 9 nM at MC4-R (average of four studies) and a Ki value of 2040 nM for MC1-R. In functional studies, the cyclic peptide of Compound 1 was determined to be a partial agonist, with intrinsic activity of 47% at MC4-R where NDP-α-MSH is 100%, and with an EC₅₀ of 2 nM (average of four studies).

In rat feeding studies, using bremelanotide as a positive control, the cyclic peptide of Compound 7 was found to reduce food intake and decrease the rate of change in body weight. Using the methods as described above, groups of 8 rats each rats received 1 mg/kg bremelanotide, 0.3 mg/kg of the cyclic peptide of Compound 7, 1 mg/kg of the cyclic peptide of Compound 7 or 3.2% mannitol control. For the 0-2, 0-4 and 0-20 hour periods, the decrease in food consumption in rats receiving 0.3 mg/kg of the cyclic peptide of Compound 7 was statistically significant compared to control, and for the 0-4, 4-20 and 0-20 hour periods, the decrease in food consumption in rats receiving 1 mg/kg of the cyclic peptide of Compound 7 was statistically significant compared to control. The 0-20 hour percent change in body weight was also statistically significant compared to control for the group receiving either 0.3 or 1 mg/kg of the cyclic peptide of Compound 7.

In rat penile erection studies, again using bremelanotide as a positive control, the cyclic peptide of Compound 7 was not found to result in a statistically significant increase in observed spontaneous erections when administered by IV means. Vehicle alone resulted in an average of 0.571±0.2 spontaneous erections per rat in one hour (n=7), the cyclic peptide of Compound 7 resulted in an average of 1.429±0.69 spontaneous erections per rat in one hour (n=7), and bremelanotide resulted in a statistically significant average of 3.167±0.79 spontaneous erections per rat in one hour (n=6). Both the cyclic peptide of Compound 7 and bremelanotide were administered at a dose of 1 mg/kg.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above are hereby incorporated by reference.

1. A cyclic peptide of formula (I): 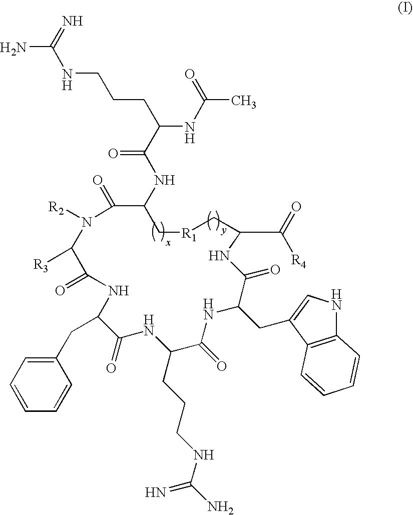
including all enantiomers, stereoisomers or diastereoisomers thereof, or a pharmaceutically acceptable salt of any of the foregoing,
wherein:
R₁ is —C(═O)—NH— or —NH—C(═O)—;
R₂ is —H or —CH₂—, wherein if R₂ is —CH₂—, then R₂ forms a pyrrolidine ring with R₃ of the structure 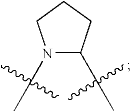
R₃ is —(CH₂)₂— 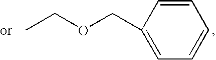
wherein if R₃ is —(CH₂)₂—, then R₃ forms a pyrrolidine ring with R₂; and
R₄ is —OH or —NH₂;
wherein
if R₁ is —C(═O)—NH—, then x is 1 and y is 4 or x is 2 and y is 3,
and if R₁ is —NH—C(═O)—, then x is 3 and y is 2.

2. The cyclic peptide of claim 1 of formula (II): 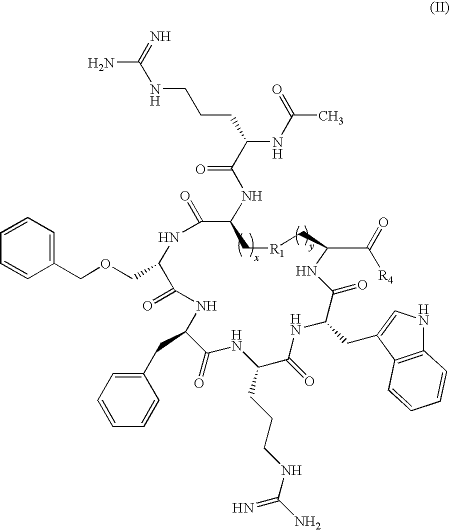
or a pharmaceutically acceptable salt thereof, wherein R₁, R₄, x and y are as defined in claim 1.

3. The cyclic peptide of claim 2 that is:
Ac-Arg-cyclo(Glu-Ser(Bzl)-D-Phe-Arg-Trp-Orn)-OH (SEQ ID NO:4);
Ac-Arg-cyclo(Orn-Ser(Bzl)-D-Phe-Arg-Trp-Glu)-OH (SEQ ID NO:5);
Ac-Arg-cyclo(Asp-Ser(Bzl)-D-Phe-Arg-Trp-Lys)-OH (SEQ ID NO:6); or
Ac-Arg-cyclo(Asp-Ser(Bzl)-D-Phe-Arg-Trp-Lys)-NH₂ (SEQ ID NO:7).

4. The cyclic peptide of claim 1 of formula (III): 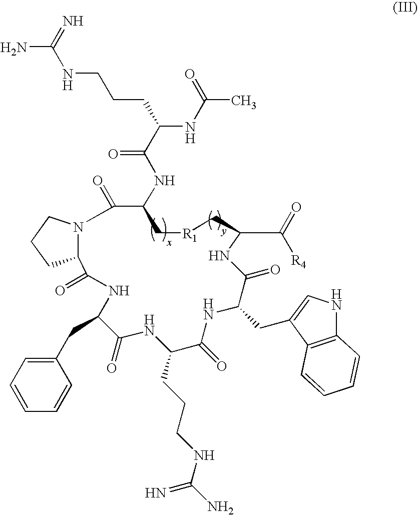
or a pharmaceutically acceptable salt thereof, wherein R₁, R₄, x and y are as defined in claim 1.

5. The cyclic peptide of claim 4 that is:
Ac-Arg-cyclo(Asp-Pro-D-Phe-Arg-Trp-Lys)-NH₂ (SEQ ID NO:8);
Ac-Arg-cyclo(Orn-Pro-D-Phe-Arg-Trp-Glu)-NH₂ (SEQ ID NO:9); or
Ac-Arg-cyclo(Asp-Pro-D-Phe-Arg-trp-Lys)-OH. (SEQ ID NO: 10).

6. A pharmaceutical composition comprising a cyclic peptide or a pharmaceutically acceptable salt thereof of claim 1 and a pharmaceutically acceptable carrier.

7. A method for treating a melanocortin receptor-mediated disease, indication, condition or syndrome in a human or non-human mammal in need thereof, comprising administering to the human or non-human mammal a therapeutically effective amount of the pharmaceutical composition of claim 6.

8. A method for treating a condition responsive to changes in melanocortin receptor function in a human or non-human mammal in need thereof, comprising administering to the human or non-human mammal a therapeutically effective amount of the pharmaceutical composition of claim 6.

9. The method of claim 8, wherein the condition is obesity, diabetes, modulation of feeding behavior or related metabolic syndrome.

10-12. (canceled)

13. A method of treating a disease, disorder and/or condition responsive to activation of the MC4 receptor in a patient in need thereof, comprising administering to the patient a therapeutically effective amount of a peptide according to claim 1.

14. A method of treating diabetes, obesity, overweight and/or diseases, disorders and/or conditions associated with obesity and/or overweight, including insulin resistance; impaired glucose tolerance; type 2 diabetes; metabolic syndrome; dyslipidemia; hyperlipidemia; hypertension; heart disorders; cardiovascular disorders; non-alcoholic fatty liver disease; joint disorders; secondary osteoarthritis; gastroesophageal reflux; sleep apnea; atherosclerosis; stroke; macro and micro vascular diseases; steatosis; gall stones; and gallbladder disorders comprising administering to a patient in need thereof a therapeutically effective amount of a peptide according to claim 1.

15. A method of reducing food intake, body weight and/or body weight gain comprising administering to an individual in need thereof a pharmacologically effective amount of a peptide according to claim 1.


SEQUENCE LISTING (13 sequences)

SEQ ID NO: 1 - protein, 7 aa, Artificial Sequence
  Synthetic melanocortin binding sequence derived from human NDP-alpha-MSH
XDHXRWK

SEQ ID NO: 2 - protein, 7 aa, Artificial Sequence
  Synthetic melanocortin binding sequence drived from human NDP-alpha-MSH
XDHXRWK

SEQ ID NO: 3 - protein, 4 aa, Artificial Sequence
  Synthetic melanocortin receptor binding peptide of the invention
HFRW

SEQ ID NO: 4 - protein, 7 aa, Artificial Sequence
  Synthetic melanocortin receptor binding peptide of the invention
REXXRWX

SEQ ID NO: 5 - protein, 7 aa, Artificial Sequence
  Synthetic melanocortin receptor binding peptide of the invention
RXXXRWE

SEQ ID NO: 6 - protein, 7 aa, Artificial Sequence
  Synthetic melanocortin receptor binding peptide of the invention
RDXXRWK

SEQ ID NO: 7 - protein, 7 aa, Artificial Sequence
  Synthetic melanocortin receptor binding peptide of the invention
RDXXRWK

SEQ ID NO: 8 - protein, 7 aa, Artificial Sequence
  Synthetic melanocortin receptor binding peptide of the invention
RDPXRWK

SEQ ID NO: 9 - protein, 7 aa, Artificial Sequence
  Synthetic melanocortin receptor binding peptide of the invention
RXPXRWE

SEQ ID NO: 10 - protein, 7 aa, Artificial Sequence
  Synthetic melanocortin receptor binding peptide of the invention
RDPXRWK

SEQ ID NO: 11 - protein, 7 aa, Artificial Sequence
  Synthetic melanocortin receptor binding peptide of the invention
RDXXRWK

SEQ ID NO: 12 - protein, 13 aa, Artificial Sequence
  Synthetic melanocortin binding sequence derived from human NDP-alpha-MSH
SYSMEHFRWGKPV

SEQ ID NO: 13 - protein, 13 aa, Artificial Sequence
  Synthetic melanocortin binding sequence derived from human NDP-alpha-MSH
SYSXEHXRWGKPV